(19) United States
(12) Patent Application Publication
(10) Pub. No.: US 2025/0002448 A1
(43) Pub. Date: Jan. 2, 2025

(54) ALLOSTERIC MODULATORS OF NICOTINIC ACETYLCHOLINE RECEPTORS

(71) Applicant: MERCK SHARP & DOHME LLC, Rahway, NJ (US)

(72) Inventors: Ian M. Bell, Lansdale, PA (US); Brendan M. Crowley, Collegeville, PA (US); James Fells, Lansdale, PA (US); Patrick Bazzini, Strasbourg (FR); Jean-Marie Contreras, Westhouse (FR); Fabrice Garrido, Strasbourg (FR); Belinda C. Huff, Camden Park (AU); Christophe Joseph, Breuschwickersheim (FR); Christophe Morice, Widensolen (FR); Dharam Paul, Klemzig (AU); Aurelie Witzel, Illkirch-Graffenstaden (FR)

(73) Assignee: MERCK SHARP & DOHME LLC, Rahway, NJ (US)

(21) Appl. No.: 18/689,149

(22) PCT Filed: Sep. 19, 2022
(86) PCT No.: PCT/US2022/043934

§ 371 Date: Mar. 5, 2024

Related U.S. Application Data
(60) Provisional application No. 63/246,588, filed on Sep. 21, 2021.

(51) Int. Cl. C07C 233/60; A61K 31/165; A61K 31/337; A61K 31/343; A61K 31/44; A61K 31/443; A61K 45/06; C07C 233/59; C07D 213/30; C07D 305/08; C07D 307/94; C07D 405/12; C07D 407/12

(52) U.S. Cl. CPC C07C 233/60; A61K 31/165; A61K 31/337; A61K 31/343; A61K 31/44; A61K 31/443; A61K 45/06; C07C 233/59; C07D 213/30; C07D 305/08; C07D 307/94; C07D 405/12; C07D 407/12; C07C 2601/14; C07C 2603/94

(57) ABSTRACT

The present disclosure relates to compounds of formula (I) that are useful as modulators of a7 nAChR, compositions comprising such compounds, and the use of such compounds for preventing, treating, or ameliorating disease, particularly disorders of the central nervous system such as cognitive impairments in Alzheimer's disease. Parkinson's disease, and schizophrenia, as well as for L-DOPA induced-dyskinesia and inflammation.

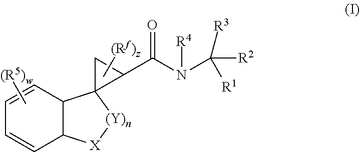

### FIELD OF THE INVENTION

The present disclosure relates to compounds that are useful as modulators of a7 nAChR, compositions comprising such compounds, and the use of such compounds for preventing, treating, or ameliorating disease, particularly disorders of the central nervous system such as cognitive impairments in Alzheimer's disease, Parkinson's disease, and schizophrenia.

### BACKGROUND OF THE INVENTION

The a7 nAChR is a fast desensitizing ligand-gated ion channel that has high permeability to Ca²⁺ In human brain, a7 nAChRs are highly expressed in the cortex and hippocampus, regions associated with cognition, see for example, Breese et al. J. Comp. Neurol. (1997) 387:385-398. In neurons, a7 nAChRs are localized in both pre-synaptic and post-synaptic structures, where activation of the receptor can modulate neurotransmitter release, neuronal excitability, and intracellular signalling, see for example, Frazier et al. J. Neurosci. (1998) 18:1187-1195.

Cognitive impairments are prevalent in many neurological and psychiatric diseases, including Alzheimer's disease (AD), schizophrenia, and Parkinson's disease, and dysfunction in cholinergic signalling contributes to the cognitive impairments of these diseases, see for example, Francis et al. J. Neurol. Neurosurg. Psychiatry (1999) 66:137-147. For example, a principal feature of the pathogenesis in AD is the loss of cholinergic neurons in the basal forebrain nuclei, whereas increasing cholinergic transmission via inhibition of acetylcholine esterase is the standard of care for the cognitive symptoms of AD. More specific to the a7 nAChR, it was recently demonstrated that encenicline, a partial agonist of the a7 nAChR, improves cognition in Alzheimer's disease, see for example, Moebius H et al., 67^{th} Annual Meeting. Am. Acad. Neurol. (AAN) 2015, Abst P7.100. Evidence implicating a7 nAChRs in the etiology of schizophrenia comes from studies demonstrating reduced expression of neuronal a7 nAChRs in the brain of schizophrenic patients and the observation that schizophrenics frequently smoke, which is believed to be a form of self-medication. In addition, variants in the promotor region of the gene coding for the a7 nAChR, CHRNA7, which impacts expression of the a7 nAChR protein, are associated with symptoms of schizophrenia, see for example, Sinkus et al. Neuropharmacology (2015) 96:274-288. Moreover, accumulating evidence from clinical trials has indicated that activating a7 nAChR with agonists may have beneficial effects on cognition, see for example, Keefe et al. Neuropsychopharmacology (2015) 40:3053-3060 and Bertrand et al. Pharmacology Reviews (2015) 67:1025-1073. Therefore, targeting the a7 nAChR represents a therapeutic strategy for the treatment of cognitive impairments associated with various cognitive disorders.

Parkinson's disease (PD) is a neurodegenerative disease characterized by progressive deficits in motor function, such as tremor, bradykinesia, rigidity and impaired postural reflex. The main pathological finding associated with the disease is degeneration of dopaminergic neurons in the substantia nigra, resulting in loss of dopaminergic tone in the striatum. L-DOPA is the current standard treatment for the motor symptoms in PD. However, chronic treatment with L-DOPA in PD patients also induces dyskinesia, a side effect of L-DOPA therapy. New lines of evidence indicate that activating a7 nAChRs acutely alleviates dyskinesia in several animal models, see for example, Zhang et al. J. Pharmacol. Exp. Ther. (2014) 351:25-32. In addition, accumulating evidence shows that pretreatment with a7 nAChR agonists may protect against neurodegeneration in nigrostriatal neurons, suggesting a7 activation may have disease modifying properties too, see for example, Suzuki et al. J. Neurosci. Res. (2013) 91:462-471. Overall, a7 nAChR is an attractive target for both ameliorating disease progression and managing dyskinesia.

In addition to its expression in the central nervous system, the a7 nAChR is widely expressed in peripheral immune cells including macrophage, monocytes, dendritic cells, and B and T cells, see for example, Rosas-Ballina et al. Science (2011) 334:98-101. Activation of peripheral a7 nAChRs is critical for inhibiting the release of proinflammatory cytokines via the cholinergic anti-inflammatory pathway, see for example, Wang et al. Nature (2003) 421:384-388. Therefore, a7 nAChR is a potential target for several inflammatory diseases such as rheumatoid arthritis, and atherosclerosis, see for example, W J de Jonge et al. British J. Pharmacol. (2007) 151:915-929.

Cough is one of the most common symptoms for which patients seek medical attention. Chronic cough, defined as a cough of greater than 8 weeks of duration, is a clinical syndrome with distinct intrinsic pathophysiology characterized by neuronal hypersensitivity.

Current treatment for chronic cough consists of antitussive therapy to decrease cough frequency or severity. However, the available antitussives have limited efficacy and their utility is further restricted by safety and abuse liabilities. Recent studies performed in healthy human volunteers indicate that activation of nAChR may represent a novel, safe, and effective antitussive strategy, see for example, Davenport et al. Pulm. Pharmacol. Ther. (2009) 22:82-89: Dicpinigaitis. Pulm. Pharmacol. Ther. (2017) 47:45-48. Furthermore, pre-clinical studies suggest that a7 nAChR is likely the target for antitussive nAChR ligands, see for example. Canning et al. Am. J. Respir. Crit. Care. Med. (2017) 195: A4498. Therefore, targeting a7 nAChR represents an attractive antitussive strategy in patients with cough.

In recent years, a7-selective positive allosteric modulators (PAMs) have been proposed as a therapeutic approach to treating cognitive impairments in AD, PD, and schizophrenia, as well as L-DOPA induced-dyskinesia, inflammation, and cough. In contrast to a7 agonists that activate the channel irrespective of endogenous agonist. PAMs increase the potency of the endogenous agonist without perturbing the temporal and spatial integrity of neurotransmission. There are two classes of a7 PAMs, type I and type II, which differ based on the functional properties of modulation. The type I PAMs (e.g. NS1738, see for example, Timmermann et al. J. Pharmacol. Exp. Ther. (2007) 323:294-307) predominantly affect the peak current with little or no effect on receptor desensitization, while the type II PAMs (e.g. PNU120596, see for example. Hurst et al. J. Neurosci. (2005) 25:4396-4405) markedly delay desensitization of the receptor. Additionally, a7 nAChR PAMs may have improved selectivity over related channel targets, presumably through binding to non-conserved regions of the receptor.

The present invention is directed to a new class of compounds that exhibit positive allosteric modulation of the a7 nAChR.

### SUMMARY OF THE INVENTION

The present disclosure relates to novel compounds of formula I and II and pharmaceutically acceptable salts thereof. These compounds may be useful, either as compounds or their pharmaceutically acceptable salts (when appropriate), in the modulation of the a7 nAChR, the prevention, treatment, or amelioration of disease, particularly disorders of the central nervous system such as cognitive impairments in Alzheimer's disease, Parkinson's disease, and schizophrenia and/or as pharmaceutical composition ingredients. As pharmaceutical composition ingredients, these compounds and their salts may be the primary active therapeutic agent, and, when appropriate, may be combined with other therapeutic agents including but not limited to acetylcholinesterase inhibitors, NMDA receptor antagonists, beta-secretase inhibitors, M4 mAChR agonists or PAMs, mGluR2 antagonists or NAMs or PAMs, 5-HT6 antagonists, histamine H3 receptor antagonists, PDE4 inhibitors, PDE9 inhibitors, HDAC6 inhibitors, antipsychotics, MAO-B inhibitors, and levodopa.

In one aspect, the present invention relates to a compound of formula I; relates to a compound of formula I:

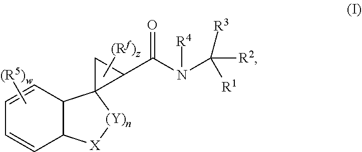

or a pharmaceutically acceptable salt thereof, wherein:
w is 0, 1, 2, 3, or 4;
n is 1, 2; 3, or 4;
z is 0, 1, 2, or 3;
q is 0, 1, or 2;
X is O, C(R^{d}R^{e}), NRⁱ, or S(O) m;
Y is C(R^{f}R^{g});
R^{d} and R^{e} are each independently selected from hydrogen, halogen, (C₁-C₄)alkyl and (C₁-C₄)haloalkyl;
each R^{f} and R^{g} is independently selected from hydrogen, halogen, (C₁-C₄)alkyl and (C₁-C₄)haloalkyl;
each R^{h} is independently selected from halogen, (C₁-C₄)alkyl and (C₁-C₄) baloalkyl; Rⁱ is selected from hydrogen. (C₁-C₄)alkyl, (C₁-C₄)haloalkyl, S(O)₂ (C₁-C₄)alkyl, and C═O(C₁-C₄)alkyl;
m is 0, 1, or 2;
each R⁵ is independently selected from halogen, cyano, hydroxy, (C₁-C₄)alkyl, (C₃-C₆)cycloalkyl, (C₁-C₄)alkoxy, aryl, heteroaryl, and heterocyclyl, wherein said alkyl, cycloalkyl, aryl, heteroaryl and heterocyclyl are optionally substituted with one or more substituents independently selected from R⁶;
R⁶ is halogen, OH, (C₁-C₄)alkyl, aryl, heteroaryl, or heterocyclyl;
R⁴ is hydrogen or (C₁-C₄)alkyl;
R¹ is hydrogen, (C₁-C₄)alkyl or (C₁-C₄) heteroalkyl;
R² and R³ are each independently selected from hydrogen, halogen, cyano, (C₁-C₈)alkyl, S(O)_{q}(C₁-C₆)alkyl, (C₁-C₈)haloalkyl, (C₁-C₄) heteroalkyl, (C₁-C₆) alkoxy, (C₁-C₆)alkoxy (C₁-C₆)alkyl, (C₁-C₆)alkylamino, amino (C₁-C₆)alkyl, hydroxy (C₁-C₆)alkyl. (C₃-C₈)cycloalkyl, (C₃-C₈)cycloalkyl(C₁-C₆) alkoxy, heterocyclyl, aryl, and heteroaryl; wherein said alkoxy, alkylamino, aminoalkyl, hydroxyalkyl, alkyl, haloalkyl, cycloalkyl, aryl, heteroaryl and heterocyclyl are optionally substituted with one or more R⁷ substituents;
each R⁷ substituent is independently selected from F, Cl, Br, OH, oxo, CF₃, OCF₃, CN, (C₁-C₆)alkyl, O(C₁-C₆)alkyl, S(O)_{q}(C₁-C₆)alkyl, C═O(C₁-C₄)alkyl, (C═O)NR⁸R⁹, (C═O)OR⁸, (C₁-C₄)alkynyl, (C₃-C₆)cycloalkyl, O(C₃-C₆)cycloalkyl, C═O(C₃-C₆)cycloalkyl, aryl, heteroaryl and heterocyclyl, wherein said alkyl, aryl, heteroaryl and heterocyclyl are independently substituted with 0, 1, 2, 3, or 4 R¹⁰ substituents: or
when R¹ is (C₁-C₄)alkyl and R² is (C₁-C₅)alkyl(C₁-C₄) heteroalkyl, or (C₁-C₈)haloalkyl, R¹ and R² together with the carbon atom to which they are attached, optionally may form a 3- to 8-membered unsaturated ring substituted with one or more R⁷ substituents;
R⁸ is H or (C₁-C₄)alkyl;
R⁹ is H or (C₁-C₄)alkyl; and
R¹⁰ is halogen. (C₁-C₅)alkyl, CF₃, OH or oxo.

The present invention also includes pharmaceutical compositions containing a compound of the present invention and methods of preparing such pharmaceutical compositions. The present invention further includes methods of preventing, treating, or ameliorating the cognitive impairments associated with Alzheimer's disease. Parkinson's disease, and schizophrenia.

Other embodiments, aspects and features of the present invention are either further described in or will be apparent from the ensuing description, examples and appended claims.

### DETAILED DESCRIPTION OF THE INVENTION

The present invention includes compounds of formula I above, and pharmaceutically acceptable salts thereof. The compounds of formula I are positive allosteric modulators of a7 nAChR.

In a first embodiment of the invention, R¹ is hydrogen, or (C₁-C₄)alkyl and the other groups are as provided in the general formula above.

In a second embodiment of the invention, R¹ is hydrogen, methyl, ethyl, or propyl and the other groups are as provided in the general formula above.

In a third embodiment of the invention. R¹ is hydrogen, or methyl and the other groups are as provide in the general formula above.

In a fourth embodiment of the invention. R² and R³ are each independently selected from halogen, (C₁-C₅)alkyl, S(O)_{q}(C₁-C₆)alkyl, (C₁-C₅)haloalkyl, (C₁-C₄) heteroalkyl, (C₁-C₆)alkoxy. (C₁-C₆)alkoxy (C₁-C₆)alkyl, (C₁-C₆)alkylamino, amino (C₁-C₆)alkyl, hydroxy (C₁-C₆)alkyl. (C₃-C₅)cycloalkyl. (C₃-C₈)cycloalkyl(C₁-C₆) alkoxy, heterocyclyl, aryl, and heteroaryl; wherein said alkoxy, alkylamino, aminoalkyl, hydroxyalkyl, alkyl, haloalkyl, cycloalkyl, aryl, heteroaryl and heterocyclyl are optionally substituted with one or more R⁷ substituents; and the other groups are provided in the general formula above, or as in the first through third embodiments.

In a fifth embodiment of the invention. R² and R³ are each independently selected from halogen. (C₁-C₅)alkyl, (C₁-C₅)haloalkyl, (C₁-C₄) heteroalkyl, (C₁-C₆) alkoxy, (C₁-C₆)alkoxy (C₁-C₆)alkyl, hydroxy (C₁-C₆)alkyl, (C₃-C₈)cycloalkyl, (C₃-C₈)cycloalkyl(C₁-C₆) alkoxy, heterocyclyl, aryl, and heteroaryl; wherein said alkoxy, hydroxyalkyl, alkyl, haloalkyl, cycloalkyl, aryl, heteroaryl and heterocyclyl are optionally substituted with one or more R⁷ substituents; and the other groups are provided in the general formula above, or as in the first through third embodiments.

In a sixth embodiment of the invention. R² is selected from halogen, (C₁-C₈)alkyl, (C₁-C₅)haloalkyl. (C₁-C₄) heteroalkyl. (C₁-C₆) alkoxy, (C₁-C₆)alkoxy (C₁-C₆)alkyl, hydroxy (C₁-C₆)alkyl, wherein said alkoxy, hydroxyalkyl, alkyl, and haloalkyl are optionally substituted with one or more R⁷ substituents; and the other groups are provided in the general formula above, or as in the first through third embodiments, and the other groups are provided in the general formula above, or as in the first through third embodiments.

In a seventh embodiment of the invention, R³ is selected from (C₁-C₈)alkyl, (C₁-C₅)haloalkyl. (C₁-C₄) heteroalkyl. (C₃-C₈)cycloalkyl. (C₃-C₈)cycloalkyl(C₁-C₆) alkoxy, heterocyclyl, aryl, and heteroaryl; wherein said alkyl, haloalkyl, cycloalkyl, aryl, heteroaryl and heterocyclyl are optionally substituted with one or more R⁷ substituents; and the other groups are provided in the general formula above, or as in the first through sixth embodiments.

In an eighth embodiment of the invention. R³ is selected from (C₃-C₈)cycloalkyl, (C₃-C₈)cycloalkyl(C₁-C₆) alkoxy, heterocyclyl, aryl, and heteroaryl; wherein said alkyl, cycloalkyl, aryl, heteroaryl and heterocyclyl are optionally substituted with one or more R⁷ substituents; and the other groups are provided in the general formula above, or as in the first through sixth embodiments.

In a ninth embodiment of the invention, R¹ is (C₁-C₄)alkyl and R² is (C₁-C₅)alkyl(C₁-C₄) heteroalkyl, or (C₁-C₅)haloalkyl and R¹ and R² together with the carbon atom to which they are attached, form a 3- to 8—membered unsaturated ring substituted with one or more R⁷ substituents; and the other groups are provided in the general formula above, or as in the first through fifth, sixth, seventh and eighth embodiments.

In a tenth embodiment of the invention, X is O or C(R^{d}R^{e}); and the other groups are provided in the general formula above, or as in the first through ninth embodiments.

In an eleventh embodiment of the invention, each R^{f} and R^{g} is independently selected from hydrogen, halogen, methyl, ethyl, propyl, trifluoromethyl, trifluoroethyl, and difluoromethyl, and the other groups are provided in the general formula above, or as in the first through tenth embodiments.

In a twelfth embodiment of the invention, each R^{d} and R^{e} is independently selected from hydrogen, halogen, methyl, ethyl, propyl, trifluormethyl, trifluoroethyl, and difluoromethyl, and the other groups are provided in the general formula above, or as in the first through eleventh embodiments.

In a thirteenth embodiment of the invention, each R⁵ is independently selected from halogen, cyano, hydroxy. (C₁-C₄)alkyl, and (C₁-C₄)alkoxy, wherein said alkyl and alkoxy are optionally substituted with one or more substituents independently selected from R⁶; and the other groups are provided in the general formula above, or as in the first through twelfth embodiments.

In a fourteenth embodiment of the invention, each R⁵ is independently selected from halogen, hydroxy, and (C₁-C₄)alkyl; and the other groups are provided in the general formula above, or as in the first through twelfth embodiments.

In a fifteenth embodiment of the invention, each R⁷ substituent is independently selected from F, Cl, OH, oxo, CF₃, OCF₃, CN, (C₁-C₆)alkyl, O(C₁-C₄)alkyl, (C₃-C₆)cycloalkyl, O(C₃-C₆)cycloalkyl. C═O(C₃-C₆)cycloalkyl, aryl, heteroaryl and heterocyclyl, wherein said alkyl, aryl, heteroaryl and heterocyclyl are independently substituted with 0, 1, 2, 3, or 4 R¹⁰ substituents; and the other groups are provided in the general formula above, or as in the first through thirteenth embodiments.

In a sixteenth embodiment of the invention, each R⁷ substituent is independently selected from F. Cl, OH. CF₃, (C₁-C₆)alkyl, and O(C₁-C₄)alkyl; and the other groups are provided in the general formula above, or as in the first through fourteenth embodiments.

Representative compounds of the present invention are as follows, where each named compound is intended to encompass its individual isomers, mixtures thereof (including racemates and diastereomeric mixtures), as well as pharmaceutically acceptable salts thereof:

The invention is also directed to a compound, or a pharmaceutically acceptable salt thereof, selected from the following exemplified compounds:
N-[(1R)-1-(4-Ethoxyphenyl)-2-methoxy-ethyl]-2′,3′-dihydrospiro[cyclopropane-1,1′-indene]-2-carboxamide;
(1R,2R)-N-[ (1R)-1-(4-Ethoxyphenyl)-2-methoxy-ethyl]-2′, 3′-dihydrospiro[cyclopropane-1,1′-indene]-2-carboxamide;
trans-5′-Fluoro-N-[(1R)-1-(4-ethoxyphenyl)-2-methoxyethyl]-2′, 3′-dihydrospiro[cyclopropane-1,1′-indene]-2-carboxamide;
cis-5-Fluoro-N-[ (1R)-1-(4-ethoxyphenyl)-2-methoxyethyl]-2H-spiro[1-benzofuran-3,1′-cyclopropane]-2′-carboxamide;
N-[3-(4-Fluorophenyl) oxetan-3-yl]-2′, 3′-dihydrospiro[cyclopropane-1,1′-indene]-2-carboxamide;
N-[(1R)-1-(4-Ethoxyphenyl)-2-methoxyethyl]-6′-fluoro-2′, 3′-dihydrospiro[cyclopropane-1,1′-indene]-2-carboxamide;
N-[(1R)-1-Cyclohexyl-2-hydroxyethyl]-6′-fluoro-2′, 3′-dihydrospiro[cyclopropane-1,1′-indene]-2-carboxamide;
N-[(1R)-1-(6-Ethoxypyridin-3-yl)-2-hydroxyethyl]-6′-fluoro-2′, 3′-dihydrospiro[cyclopropane-1,1′-indene]-2-carboxamide:
(6′-Fluoro-N-[3-(4-fluorophenyl) oxetan-3-yl]-2′, 3′-dihydrospiro[cyclopropane-1,1′-indene]-2-carboxamide;
N-[(1R)-1-Cyclohexyl-2,2-difluoroethyl]-6′-fluoro-2′, 3′-dihydrospiro[cyclopropane-1,1′-indene]-2-carboxamide;
(N-[(1R)-1-Cyclohexyl-2-hydroxyethyl]-2H-spiro[1-benzofuran-3,1′-cyclopropane]-2′-carboxamide;
N-[3-(4-Fluorophenyl) oxetan-3-yl]-2H-spiro[1-benzofuran-3,1′-cyclopropane]-2′-carboxamide; cis-N-[(1R)-1-(4-Ethoxyphenyl)-2-methoxyethyl]-5-fluoro-2H-spiro[I-benzofuran-3,1′-cyclopropane]-2′-carboxamide:
cis-N-[ (1R)-1-(6-Ethoxypyridin-3-yl)-2-hydroxyethyl]-5-fluoro-2H-spiro[1-benzofuran-3,1′-cyclopropane]-2′-carboxamide;
cis-N-[(1R)-1-(6-Ethoxypyridin-3-yl)-2,2-difluoroethyl]-5-fluoro-2H-spiro[1-benzofuran-3,1′-cyclopropane]-2′-carboxamide;
cis-N-[(1R)-1-(6-Ethoxypyridin-3-yl)-2-fluoroethyl]-5-fluoro-2H-spiro[1-benzofuran-3,1′-cyclopropane]-2′-carboxamide:
cis-N-[(1R)-1-Cyclohexyl-2,2-difluoroethyl]-5-fluoro-2H-spiro[1-benzofuran-3,1′-cyclopropane]-2′-carboxamide;
cis-N-[ (1R)-1-Cyclohexyl-2-hydroxyethyl]-5-fluoro-2H-spiro[1-benzofuran-3,1′-cyclopropane]-2′-carboxamide:
cis-5-Fluoro-N-[1-(6-methoxypyridin-3-yl)cyclopropyl]-2H-spiro[1-benzofuran-3,1′-cyclopropane]-2′-carboxamide.
cis-S-Fluoro-N-[1-(2-methoxypyridin-4-vl)cyclopropyl]-2H-spiro[1-benzofuran-3,1′-cyclopropane]-2′-carboxamide; and
trans-N-[(1R)-1-(4-Ethoxyphenyl)-2-methoxyethyl]-3′,4′-dihydro-2′H-spiro[cyclopropane-1,1′-naphthalene]-2-carboxamide.

Other embodiments of the present invention include the following:
(a) A pharmaceutical composition comprising a compound of formula I and a pharmaceutically acceptable carrier.
(b) The pharmaceutical composition of (a), further comprising a second therapeutic agent selected from the group consisting of acetylcholinesterase inhibitors such as donepezil, rivastigmine, and galantamine: NMDA receptor antagonists such as memantine; beta-secretase inhibitors such as verubecestat, and AZD3293; M4 mAChR agonists or PAMs; mGluR2 antagonists or NAMs or PAMs: 5-HT6 antagonists such as idalopirdine, RVT-101, AVN-101. AVN322, SUVN-502, and SYN-120; histamine H3 receptor antagonists such as S38093; PDE4 inhibitors such as HT0712: PDE9 inhibitors such as BI40936: HDAC6 inhibitors; antipsychotics: LRRK2 inhibitors: MAO-B inhibitors; and levodopa.
(c) The pharmaceutical composition of (b), wherein the second therapeutic agent is an antipsychotic selected from the group consisting of clozapine, olanzapine, risperidone, aripiprazole, quetiapine, haloperidol, loxapine, thioridazine, molindone, thiothixene, fluphenazine, mesoridazine, trifluoperazine, chlorpromazine, and perphenazine.
(d) A pharmaceutical combination that is (i) a compound of formula I and (ii) a second therapeutic agent selected from the group consisting of acetylcholinesterase inhibitors such as donepezil, rivastigmine, and galantamine: NMDA receptor antagonists such as memantine: beta-secretase inhibitors such as verubecestat, and AZD3293: M4 mAChR agonists or PAMs, mGluR2 antagonists or NAMs or PAMs: 5-HT6 antagonists such as idalopirdine, RVT-101, AVN-101. AVN322, SUVN-502, and SYN-120; histamine H3 receptor antagonists such as S38093: PDE4 inhibitors such as HT0712; PDE9 inhibitors such as BI40936: HDAC6 inhibitors: antipsychotics; LRRK2 inhibitors: MAO-B inhibitors; and levodopa wherein the compound of formula I and the second therapeutic agent are each employed in an amount that renders the combination effective for treating cognitive impairments associated with Alzheimer's disease, Parkinson's disease, or schizophrenia.
(e) The combination of (d), wherein the second therapeutic agent is an antipsychotic selected from the group consisting of clozapine, olanzapine, risperidone, aripiprazole, quetiapine, haloperidol, loxapine, thioridazine, molindone, thiothixene, fluphenazine, mesoridazine, trifluoperazine, chlorpromazine, and perphenazine.
(f) A use of a compound of formula I in the preparation of a medicament for modulating a7 nAChR activity in a subject in need thereof.
(g) A use of a compound of formula I in the preparation of a medicament for treating cognitive impairments associated with Alzheimer's disease, Parkinson's disease, and schizophrenia in a subject in need thereof.
(h) A method of treating cognitive impairments associated with Alzheimer's disease, Parkinson's disease, and schizophrenia and/or reducing the likelihood or severity of symptoms of cognitive impairments associated with Alzheimer's disease, Parkinson's disease, and schizophrenia in a subject in need thereof, which comprises administering to the subject an effective amount of a compound of formula I.
(i) The method of (h), wherein the compound of formula I is administered in combination with an effective amount of at least one second therapeutic agent selected from the group consisting of acetylcholinesterase inhibitors such as donepezil, rivastigmine, and galantamine: NMDA receptor antagonists such as memantine; beta-secretase inhibitors such as verubecestat, and AZD3293: M4 mAChR agonists or PAMs, mGluR2 antagonists or NAMs or PAMs; 5-HT6 antagonists such as idalopirdine, RVT-101. AVN-101, AVN322. SUVN-502, and SYN-120: histamine H3 receptor antagonists such as S38093: PDE4 inhibitors such as HT0712; PDE9 inhibitors such as BI40936: HDAC6 inhibitors: antipsychotics; LRRK2 inhibitors: MAO-B inhibitors; and levodopa.
j) The method of (i), wherein the second therapeutic agent is an antipsychotic selected from the group consisting of clozapine, olanzapine, risperidone, aripiprazole, quetiapine, haloperidol, loxapine, thioridazine, molindone, thiothixene, fluphenazine, mesoridazine, trifluoperazine, chlorpromazine, and perphenazine.
(k) A method of modulating a7 nAChR activity in a subject in need thereof, which comprises administering to the subject the pharmaceutical composition of (a), (b), or (c) or the combination of (d) or (e).
(l) A method of treating cognitive impairments associated with Alzheimer's disease, Parkinson's disease, and schizophrenia and/or reducing the likelihood or severity of symptoms of cognitive impairments associated with Alzheimer's disease, Parkinson's disease, and schizophrenia in a subject in need thereof, which comprises administering to the subject the pharmaceutical composition of (a), (b), or (c) or the combination of (d) or (e).

In the embodiments of the compounds and salts provided above, it is to be understood that each embodiment may be combined with one or more other embodiments, to the extent that such a combination provides a stable compound or salt and is consistent with the description of the embodiments. It is further to be understood that the embodiments of compositions and methods provided as (a) through (1) above are understood to include all embodiments of the compounds and/or salts, including such embodiments as result from combinations of embodiments.

Additional embodiments of the invention include the pharmaceutical compositions, combinations, uses and methods set forth in (a) through (1) above, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate.

The present invention also includes a compound of the present invention for use (i) in. (ii) as a medicament for, or (iii) in the preparation of a medicament for: (a) preventing or treating cognitive impairments associated with Alzheimer's disease. Parkinson's disease, schizophrenia, and L-DOPA induced-dyskinesia, or (b) treating cognitive impairments associated with Alzheimer's disease, Parkinson's disease, schizophrenia, and L-DOPA induced-dyskinesia and/or reducing the likelihood or severity of symptoms of cognitive impairments associated with Alzheimer's disease, Parkinson's disease, schizophrenia and L-DOPA induced-dyskinesia, or (c) use in medicine. In these uses, the compounds of the present invention can optionally be employed in combination with one or more second therapeutic agents selected from acetylcholinesterase inhibitors such as donepezil, rivastigmine, and galantamine; NMDA receptor antagonists such as memantine: beta-secretase inhibitors such as verubecestat, and AZD3293; M4 mAChR agonists or PAMs: mGluR2 antagonists or NAMs or PAMs, 5-HT6 antagonists such as idalopirdine, RVT-101, AVN-101, AVN322, SUVN-502, and SYN-120; histamine H3 receptor antagonists such as S38093: PDE4 inhibitors such as HT0712; PDE9 inhibitors such as BI40936; HDAC6 inhibitors: antipsychotics; LRRK2 inhibitors; MAO-B inhibitors; and levodopa.

Chemical names, common names, and chemical structures may be used interchangeably to describe the same structure. If a chemical compound is referred to using both a chemical structure and a chemical name and an ambiguity exists between the structure and the name, the structure is understood to predominate.

As used herein, the term “5-membered heteroaryl ring” refers to a stable unsaturated 5-membered ring that contains from 1 to 4 heteroatoms selected from the group consisting of O. N, and S. A 5-membered heteroaryl ring within the scope of this definition includes but is not limited to: furanyl, imidazolyl, isothiazolyl, isoxazolyl, oxadiazolyl, oxazolyl, pyrazolyl, pyrrolyl, tetrazolyl, thiadiazolyl, thiazolyl, thienyl, and triazolyl.

In another embodiment. “5-membered heteroaryl ring” is furanyl, imidazolyl, isothiazolyl, isoxazolyl, oxadiazolyl, oxazolyl, pyrazolyl, pyrrolyl, tetrazolyl, thiadiazolyl, thiazolyl, thienyl, and triazolyl.

As used herein, the term “6-membered heteroaryl ring” refers to a stable unsaturated 6-membered ring that contains from 1 to 4 heteroatoms selected from the group consisting of O, N, and S. A 6-membered heteroaryl ring within the scope of this definition includes but is not limited to: pyridazinyl, pyridyl, and pyrimidyl.

As used herein, the term “administration” and variants thereof (e.g., “administering” a compound) in reference to a compound of the invention means providing the compound to the individual in need of treatment. When a compound of the invention is provided in combination with one or more other active agents (e g., cholinesterase inhibitors such as donepezil, rivastigmine, and galantamine). “administration” and its variants are each understood to include concurrent and sequential administration of the compound or salt and other agents. The term “alkenyl” refers to a hydrocarbon radical straight or branched containing from 2 to 12 carbon atoms and at least one carbon to carbon double bond. Up to four carbon-carbon double bonds may be present. Thus, “C₂-C₆ alkenyl” means an alkenyl radical having from 2 to 6 carbon atoms. Thus, “C₂-C₄ alkenyl” means an alkenyl radical having from 2 to 4 carbon atoms. Alkenyl groups include ethenyl, propenyl, butenyl. 3-methylbutenyl and so on. In one embodiment, an alkenyl group is linear. In another embodiment, an alkenyl group is branched.

The term “alkyl” refers to an aliphatic hydrocarbon group having one of its hydrogen atoms replaced with a bond. An alkyl group may be straight or branched. An alkyl group contains from 1 to 8 carbon atoms[(C₁-C₈)alkyl] or from 1 to 6 carbon atoms[(C₁-C₆)alkyl] or from 1 to 4 carbon atoms[(C₁-C₄)alkyl]. Non-limiting examples of alkyl groups include methyl (Me), ethyl, n-propyl, isopropyl, n-butyl, sec-butyl, isobutyl, and tert-butyl. In one embodiment, an alkyl group is linear. In another embodiment, an alkyl group is branched. When “alkyl” is substituted, said “alkyl” includes alkyl, O-alkyl, S-alkyl and (C═O)-alkyl.

The term “alkynyl” refers to a hydrocarbon radical straight or branched containing from 2 to 12 carbon atoms and at least one carbon to carbon triple bond. Up to three carbon-carbon triple bonds may be present. Thus, “C₂-C₆ alkynyl” means an alkynyl radical having from 2 to 6 carbon atoms. Thus, “C₂-C₄ alkynyl” means an alkynyl radical having from 2 to 4 carbon atoms. Alkynyl groups include ethynyl, propynyl, butynyl, 3-methylbutynyl and so on. In one embodiment, an alkynyl group is linear. In another embodiment, an alkynyl group is branched.

The term “alkoxy” refers to an alkyl(carbon and hydrogen chain) group singularly bonded to oxygen (R—O). Non-limiting examples of alkoxy are methoxy (CH₃O—), ethoxy (CH₃CH₂O—) and butoxy (CH₃CH₂CH₂O—).

The term “aryl” refers to any mono- and poly-carbocyclic ring systems wherein the individual carbocyclic rings in the polyring systems are fused or attached to each other via a single bond and wherein at least one ring is aromatic. Suitable aryl groups include phenyl, indanyl, naphthyl, tetrahydronaphthyl, and biphenyl. Aryl ring systems may include, where appropriate, an indication of the variable to which a particular ring atom is attached. Unless otherwise indicated, substituents to the aryl ring systems can be attached to any ring atom, provided that such attachment results in formation of a stable ring system.

In an embodiment, “aryl” is phenyl.

When “aryl” is substituted, said “aryl” includes aryl and O)-aryl. “Celitek” (Fluka)diatomite is diatomaceous earth, and can be referred to as “celite”.

The term “compound” refers to the free compound and, to the extent they are stable, any hydrate or solvate thereof. A hydrate is the compound complexed with water, and solvate is the compound complexed with an organic solvent.

The term “composition” is intended to encompass a product comprising the specified ingredients, as well as any product which results from combining the specified ingredients.

The term “cycloalkyl” as used herein, refers to any non-aromatic mono- and poly-carbocyclic ring systems comprising from 3 to 10 ring carbon atoms[(C₃-C₁₀)cycloalkyl], or from 3 to 6 ring carbon atoms[(C₃-C₆)cycloalkyl] wherein the individual carbocyclic rings in the polyring systems are fused, including spiro ring fusions, or attached to each other via a single bond. Non-limiting examples of monocyclic cycloalkyls include cyclopropyl, cyclobutyl, cyclopentyl, cyclohexyl, bicyclo[4.1.0]heptyl, spiro[2.4]heptyl, spiro[3.3]heptyl, spiro[2.5]octyl, and cycloheptyl. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

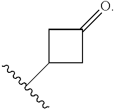 [SMILES: *C1CC(=O)C1]

When “cycloalkyl” is substituted, said “cycloalkyl” includes cycloalkyl, O-cycloalkyl and (C═O))-cycloalkyl.

In an embodiment, “cycloalkyl” is cyclopropyl, cyclobutyl, cyclopentyl or cyclohexyl.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of one or more symptoms of the disease or condition being treated. In another embodiment, the effective amount is a “prophylactically effective amount” for reduction of the severity or likelihood of one or more symptoms of the disease or condition. The term also includes herein the amount of active compound sufficient to modulate a7 nAChR activity and thereby elicit the response being sought (i.e., a “therapeutically effective amount”). When the active compound (i.e., active ingredient) is administered as the salt, references to the amount of active ingredient are to the free acid or free base form of the compound.

The term “halogen” (or “halo”) refers to atoms of fluorine, chlorine, bromine and iodine (alternatively referred to as fluoro (F), chloro(Cl), bromo (Br), and iodo (I)).

“Haloalkyl” refers to an alkyl group as described above wherein one or more (in particular 1 to 5) hydrogen atoms have been replaced by halogen atoms, with up to complete substitution of all hydrogen atoms with halo groups. C₁₋₆ haloalkyl, for example, includes-CF₃, —CF₂CF₃, —CHFCH₃, and the like.

The term “heteroalkyl” refers to an alkyl group where one or more of the carbon atoms is substituted by a heteroatom independently selected from N, O, or S.

“Hydroxyalkyl” refers to an alkyl group as described above in which one or more (in particular 1 to 3) hydrogen atoms have been replaced by hydroxy groups. Examples include CH₂OH, CH₂CHOH and CHOHCH₃.

The term “heteroaryl” as used herein, refers to any monocyclic or multicyclic ring system comprising 5 to 14 ring atoms, wherein from 1 to 4 of the ring atoms is independently O, N, or S and the remaining ring atoms are carbon atoms, and wherein at least one ring is aromatic. In one embodiment, a heteroaryl group has 5 to 10 ring atoms. In another embodiment, a heteroaryl group is monocyclic and has 5 or 6 ring atoms. In another embodiment, a heteroaryl group is bicyclic and has 9 or 10 ring atoms. A heteroaryl group is usually joined via a ring carbon atom but may be joined via a non-carbon atom provided that this results in a stable compound, and any nitrogen atom of a heteroaryl can be optionally oxidized to the corresponding N-oxide. The term “heteroaryl” also encompasses a heteroaryl group, as defined above, which is fused to a benzene ring. The term “heteroaryl” also encompasses any fused polycyclic ring system containing at least one ring heteroatom selected from N, O, and S, wherein at least one ring of the fused polycyclic ring system is aromatic. For example, the term “9 to 10-membered bicyclic heteroaryl” encompasses a non-aromatic 5 membered heterocyclic ring that is fused to a benzene or pyridyl ring. Non-limiting examples of heteroaryls include benzimidazolyl, benzimidazolonyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, dihydrobenzoimidazolyl, dihydrobenzofuranyl, dihydrobenzothiophenyl, dihydrobenzoxazolyl, dihydroindolyl, dihydroquinolinyl, methylenedioxy benzoyl and the like, and all isomeric forms thereof. The term “heteroaryl” also refers to partially saturated heteroaryl moieties such as, for example, tetrahydroisoquinolyl, tetrahydroquinolyl and the like, provided that they contain at least one aromatic ring. In one embodiment, a heteroaryl group is a 5-membered heteroaryl. In another embodiment, a heteroaryl group is a 6-membered heteroaryl. In another embodiment, a heteroaryl group comprises a 5- to 6-membered heteroaryl group fused to a benzene ring

In an embodiment. “heteroaryl” is benzimidazolyl, benzimidazolonyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, or triazolyl.

In another embodiment, “heteroaryl” is carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, or triazolyl.

In another embodiment. “heteroaryl” is furanyl, imidazolyl, indolinyl, indolyl, indolazinyl, indazolyl, isoindolyl, isoquinolyl, isothiazolyl, isoxazolyl, oxadiazolyl, oxazolyl, pyrazinyl, pyrazolyl, pyridazinyl, pyridazinyl, pyridyl, pyrimidyl, pyrrolyl, thiadiazolyl, thiazolyl, thienyl, or triazolyl.

The term “heterocycle” or “heterocyclyl” as used herein is intended to mean a 3- to 10-membered non-aromatic heterocycle containing from 1 to 4 heteroatoms selected from the group consisting of O. N, and S, and includes monocyclic or bicyclic groups (fused, bridged or spirocyclic). Further examples of “heterocyclyl” include, but are not limited to the following: oxazoline, isoxazoline, oxetanyl, tetrahydropyranyl, azetidinyl, 1,4-dioxanyl, hexahydroazepinyl, piperazinyl, piperidinyl, pyrrolidinyl, morpholinyl, thiomorpholinyl, dihydrofuranyl, dihydroimidazolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydrotetrazolyl, dihydrothiadiazolyl, dihydrothiazolyl, dihydrothienyl, dihydrotriazolyl, tetrahydrofuranyl, and tetrahydrothienyl, and N-oxides thereof. Attachment of a heterocyclyl substituent can occur via a carbon atom or via a heteroatom.

In an embodiment. “heterocycle” or “heterocyclyl” is oxazoline, isoxazoline, oxetanyl, tetrahydropyranyl, azetidinyl, 1,4-dioxanyl, hexahydroazepinyl, piperazinyl, piperidinyl, pyrrolidinyl, morpholinyl, or thiomorpholinyl.

In an embodiment, “heterocycle” or heterocyclyl” is dihydrofuranyl, dihydroimidazolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydrotetrazolyl, dihydrothiadiazolyl, dihydrothiazolyl, dihydrothienyl, dihydrotriazolyl, tetrahydrofuranyl, or tetrahydrothienyl.

In an embodiment. “heterocycle” or heterocyclyl” is morpholinyl and piperidinyl.

By “pharmaceutically acceptable” is meant that the ingredients of the pharmaceutical composition must be compatible with each other and not deleterious to the recipient thereof.

As used herein, the term “optionally” means that the subsequently described event(s) may or may not occur, and includes both event(s), which occur, and events that do not occur.

The term “preventing” as used herein with respect to Alzheimer's disease or other neurological diseases, refers to reducing the likelihood of disease progression.

The term “subject” (alternatively referred to herein as “patient”), as used herein, refers to an animal, preferably a mammal, most preferably a human.

The term “substituted” means that one or more hydrogens on the designated atom is replaced with a selection from the indicated group, provided that the designated atom's normal valency under the existing circumstances is not exceeded, and that the substitution results in a stable compound. Unless expressly stated to the contrary, substitution by a named substituent is permitted on any atom provided such substitution is chemically allowed and results in a stable compound. Combinations of substituents and/or variables are permissible only if such combinations result in stable compounds.

The term “substituted with one or more” refers to substitution with the named substituent or substuents, multiple degrees of substitution, up to replacing all hydrogen atams with the same or different substituents, being allowed unless the number of substituents is explicitly stated. Where the number of substituents is not explicitly stated, one or more is intended.

As used herein, “a compound of the invention” means a compound of formula I or a salt, solvate or physiologically functional derivative thereof.

The term “solvate” refers to a complex of variable stoichiometry formed by a solute (in this invention, a compound of formula I, or a salt thereof) and a solvent. Such solvents for the purpose of the invention may not interfere with the biological activity of the solute. Examples of suitable solvents include, but are not limited to, water, acetone, methanol, ethanol and acetic acid. Preferably the solvent used is a pharmaceutically acceptable solvent. Examples of suitable pharmaceutically acceptable solvents included water, ethanol and acetic acid.

A “stable” compound is a compound that can be prepared and isolated and whose structure and properties remain or can be caused to remain essentially unchanged for a period of time sufficient to allow use of the compound for the purposes described herein (e.g., therapeutic or prophylactic administration to a subject).

In one embodiment of formula I, w is 0, 1, 2, or 3.

In another embodiment of formula I, w is 0, 1, or 2.

In another embodiment of formula I, w is 0 or 1.

In another embodiment of formula I, n is 1, 2, or 3.

In another embodiment of formula I, n is 1 or 2.

In another embodiment of formula I, n is 1.

In another embodiment of formula I, z is 0, 1, or 2.

In another embodiment of formula I, z is 0 or 1.

In another embodiment of formula I, z is 0.

In another embodiment of formula I, q is 0, 1, or 2.

In another embodiment of formula I, q is 0 or 1.

In another embodiment of formula I, q is 0.

In another embodiment of formula I, m is 1, or 2.

In another embodiment of formula I, m is 0.

In another embodiment of formula I, m is 2.

In one embodiment of formula I, X is S(O)).

In another embodiment of formula I, X is O.

In another embodiment of formula I, X is O.

In another embodiment of formula I, X is S(O).

In another embodiment of formula I, X is NH.

In another embodiment of formula I, X is CR^{f}R^{g}.

In another embodiment of formula I, X is CH₂.

In one embodiment of formula I, R⁶ is halogen, OH, or (C₁-C₄)alkyl.

In another embodiment of formula I, R⁶ is halogen, aryl, heteroaryl, or heterocyclyl.

In one embodiment of formula I, R⁴ is hydrogen.

In another embodiment of formula I, R⁴ is (C₁-C₄)alkyl.

In one embodiment of formula I, R⁸ is H.

In one embodiment of formula I, R⁹ is (C₁-C₄)alkyl.

In one embodiment of formula I, each R^{d} and R^{e} is independently selected from hydrogen, fluoro, methyl, ethyl, and propyl.

In another embodiment of formula I, each R^{d} and R^{e} is independently hydrogen or fluoro.

In one embodiment of formula I, each R^{f} and R^{g} is independently selected from halogen, methyl, ethyl, and propyl.

In one embodiment of formula I, each R⁵ is independently selected from halogen, and (C₁-C₄)alkyl.

In another embodiment of formula I, R⁵ is halogen.

In one embodiment of formula I, R⁴ is hydrogen, methyl, ethyl or propyl.

In another embodiment of formula I, R⁴ is hydrogen.

In one embodiment of formula I, R⁸ is hydrogen.

In one embodiment of formula I, R⁹ is hydrogen.

In another embodiment of formula I, R¹⁰ is halogen, (C₁-C₄)alkyl, or OH.

In another embodiment of formula I. R¹⁰ is halogen or OH.

In one embodiment of formula I, R² and R³ are each independently selected from hydrogen, halogen, cyano, —S(O)_{q}(C₁-C₆)alkyl, (C₁-C₈)alkyl, (C₁-C₅)haloalkyl. (C₁-C₄) heteroalkyl, (C₁-C₆) alkoxy, (C₁-C₆)alkoxy (C₁-C₆)alkyl, (C₁-C₆)alkylamino, amino (C₁-C₆)alkyl, hydroxy (C₁-C₆)alkyl, cyclopropyl (C₁-C₆) alkoxy, cyclobutyl(C₁-C₆) alkoxy, cyclopentyl(C₁-C₆) alkoxy, cyclohexyl(C₁-C₆) alkoxy, cyclopropyl, cyclobutyl, cyclopentyl, cyclohexyl, phenyl, indanyl, tetrahydronaphthalenyl, pyrazolyl, pyridinyl, oxazolyl, oxadiazolyl, isoxazolyl, tetrahydrocyclopentapyrazolyl, imidazopyridinyl, indolyl, tetrahydropyrazolopyridinyl, dihydropyrrolopyrazolyl, pyridazinyl, pyrazolothiazolyl, piperazinyl, morpholinyl, and piperidinyl, wherein said each R² and R³ is optionally substituted with one or more R⁷ substituents, each R⁷ substituent independently selected from F, Cl, Br. OH, OXO, CF₃, OCF₃, CN, (C₁-C₆)alkyl, O(C₁-C₄)alkyl, S(O)_{q}(C₁-C₄)alkyl, C═O(C₁-C₄)alkyl, (C═O)NR⁵R⁶, (C═O)OR⁵, (C₁-C₄)alkynyl, (C₃-C₆)cycloalkyl, O(C₃-C₆)cycloalkyl, C═O(C₃—Ce)cycloalkyl, phenyl, indanyl, tetrahydronaphthalenyl, pyrazolyl, pyridinyl, oxazolyl, oxadiazolyl, isoxazolyl, tetrahydrocyclopentapyrazolyl, imidazopyridinyl, indolyl, tetrahydropyrazolopyridinyl, dihydropyrrolopyrazolyl, pyridazinyl, pyrazolothiazolyl, piperazinyl, morpholinyl, and piperidinyl, wherein R⁷ is substituted with one or more R¹⁰ selected from halogen, (C₁-C₄)alkyl, CF₃. OH and oxo.

In one embodiment of formula I, R² and R³ are each independently selected from halogen, (C₁-C₅)alkyl, (C₁-C₄)haloalkyl, (C₁-C₄) heteroalkyl, (C₁-C₆) alkoxy, (C₁-C₆)alkoxy (C₁-C₆)alkyl, hydroxy (C₁-C₆)alkyl, cyclopropyl (C₁-C₆) alkoxy, cyclobutyl(C₁-C₆) alkoxy, cyclopentyl(C₁-C₆) alkoxy, cyclohexyl(C₁-C₆) alkoxy, cyclopropyl, cyclobutyl, cyclopentyl, cyclohexyl, phenyl, pyridinyl, oxazolyl, oxadiazolyl, isoxazolyl, tetrahydrocyclopentapyrazolyl, imidazopyridinyl, indolyl, tetrahydropyrazolopyridinyl, dihydropyrrolopyrazolyl, pyridazinyl, pyrazolothiazolyl, piperazinyl, morpholinyl, and piperidinyl, wherein said each R² and R³ is optionally substituted with one or more R⁷ substituents, each R⁷ substituent independently selected from F, Cl, OCF₃, (C₁-C₆)alkyl, O(C₁-C₄)alkyl. (C₁-C₄)alkynyl, (C₃-C₆)cycloalkyl, O(C₃-C₆)cycloalkyl, wherein R⁷ is substituted with one or more R¹⁰ selected from halogen, (C₁-C₄)alkyl, CF₃, and OH.

In one embodiment of formula I, R² is selected from methoxy, hydroxymethyl, difluoromethyl, methoxymethyl, trifluoromethyl, and fluoromethyl.

In one embodiment of formula I, R³ is selected from phenyl, cyclohexyl, and pyridinyl, wherein said R³ is optionally substituted with one or more R⁷ substituents, each R⁷ substituent independently selected from F, C₁, OCF₃, (C₁-C₆)alkyl, and O(C₁-C₄)alkyl.

In one embodiment of formula I, R¹ and R² together with the carbon atom to which they are attached form an oxetanyl group.

In one embodiment of formula I, R¹ and R² together with the carbon atom to which they are attached form an cyclopropyl group.

In the compounds of formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of formula I. For example, different isotopic forms of hydrogen (H) include protium (¹H) and deuterium (²H or D). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within formula I can be prepared without undue experimentation by conventional techniques well known to those skilled in the art or by processes analogous to those described in the Schemes and Examples herein using appropriate isotopically-enriched reagents and/or intermediates.

Unless expressly stated to the contrary, all ranges cited herein are inclusive. For example, a heteroaryl ring described as containing from “1 to 3 heteroatoms” means the ring can contain 1, 2, or 3 heteroatoms. It is also to be understood that any range cited herein includes within its scope all of the sub-ranges within that range. The oxidized forms of the heteroatoms N and S are also included within the scope of the present invention.

It is understood by one skilled in the art that carbon atoms in organic molecules may often be replaced by silicon atoms to give analogous stable compounds. For example, carbon atoms in alkyl, cycloalkyl, aryl, heteroaryl, and heterocyclyl, groups may often be replaced by silicon atoms to provide stable compounds. All such compounds are within the scope of the present invention.

When any variable (for example. R) occurs more than one time in any constituent or in formula I or in any other formula depicting and describing compounds of the invention, its definition on each occurrence is independent of its definition at every other occurrence. Also, combinations of substituents and/or variables are permissible only if such combinations result in stable compounds.

The compounds of formula I may contain asymmetric or chiral centers, and, therefore, exist in different stereoisomeric forms. It is intended that all stereoisomeric forms of the compounds of formula I as well as mixtures thereof, including racemic mixtures, form part of the present invention. Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by chromatography employing columns with a chiral stationary phase. Also, some of the compounds of formula I may be atropisomers (e.g., substituted biaryls) and are considered as part of this invention.

Certain of the compounds of the present invention can exist as tautomers. For the purposes of the present invention a reference to a compound of formula I is a reference to the compound per se, or to any one of its tautomers per se, or to mixtures of two or more tautomers.

The compounds of formula I may have the ability to crystallize in more than one form, a characteristic known as polymorphism, and it is understood that such polymorphic forms (“polymorphs”) are within the scope of formula I. Polymorphism generally can occur as a response to changes in temperature or pressure or both and can also result from variations in the crystallization process. Polymorphs can be distinguished by various physical characteristics known in the art such as x-ray diffraction patterns, solubility and melting point

The invention includes within its scope all possible stoichiometric and non-stoichiometric forms of the compounds of formula I.

In the compounds of formula I, the atoms may exhibit their natural isotopic abundances, or one or more of the atoms may be artificially enriched in a particular isotope having the same atomic number, but an atomic mass or mass number different from the atomic mass or mass number predominately found in nature. The present invention is meant to include all suitable isotopic variations of the compounds of generic formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic formula I can be prepared without undue experimentation by conventional techniques well known to those skilled in the art or by processes analogous to those described in the Schemes and Examples herein using appropriate isotopically-enriched reagents and/or intermediates.

The compounds of the present invention may have utility in preventing, treating, or ameliorating Alzheimer's disease. The compounds may also be useful in preventing, treating, or ameliorating other diseases mediated by the a7 nAChR, such as schizophrenia, sleep disorders, Parkinson's disease, autism, microdeletion syndrome, inflammatory diseases, pain disorders (including acute pain, inflammatory pain and neuropathic pain) and cognitive disorders (including mild cognitive impairment). Other conditions that may be prevented, treated, or ameliorated by the compounds of the invention include pulmonary hypertension, chronic obstructive pulmonary disease (COPD), asthma, urinary incontinence, glaucoma, Trisomy 21 (Down Syndrome), cerebral amyloid angiopathy, degenerative dementia, Hereditary Cerebral Hemorrhage with Amyloidosis of the Dutch-Type (HCHWA-D), Creutzfeld-Jakob disease, prion disorders, amyotrophic lateral sclerosis, progressive supranuclear palsy, bead trauma, stroke, pancreatitis, inclusion body myositis, other peripheral amyloidoses, diabetes, kidney diseases, cancer, and atherosclerosis.

In preferred embodiments, the compounds of the invention may be useful in preventing, treating, or ameliorating Alzheimer's disease, cognitive disorders, schizophrenia, pain disorders and sleep disorders. For example, the compounds may be useful for the prevention of dementia of the Alzheimer's type, as well as for the treatment of early stage, intermediate stage or late stage dementia of the Alzheimer's type.

Potential schizophrenia conditions or disorders for which the compounds of the invention may be useful include one or more of the following conditions or diseases; schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anaesthetics, amphetamine and other psychostimulants and cocaine) psychosispsychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both the positive and the negative symptoms of schizophrenia and other psychoses: cognitive disorders including dementia (associated with Alzheimer's disease, ischemia, multi-infarct dementia, trauma, vascular problems or stroke. HIV disease, Parkinson's disease. Huntington's disease, Pick's disease, Creutzfeldt-Jacob disease, perinatal hypoxia, other general medical conditions or substance abuse): delirium, amnestic disorders or age related cognitive decline.

Thus, in another specific embodiment, the present invention provides a method for preventing, treating, or ameliorating schizophrenia or psychosis comprising administering to a patient in need thereof an effective amount of a compound of the present invention. At present, the text revision of the fourth edition of the Diagnostic and Statistical Manual of Mental Disorders (DSM-IV-TR) (2000, American Psychiatric Association, Washington DC) provides a diagnostic tool that includes paranoid, disorganized, catatonic or undifferentiated schizophrenia and substance-induced psychotic disorder. As used herein, the term “schizophrenia or psychosis” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

Potential sleep conditions or disorders for which the compounds of the invention may be useful include enhancing sleep quality: improving sleep quality: augmenting sleep maintenance: increasing the value which is calculated from the time that a subject sleeps divided by the time that a subject is attempting to sleep: decreasing sleep latency or onset (the time it takes to fall asleep): decreasing difficulties in falling asleep: increasing sleep continuity; decreasing the number of awakenings during sleep: decreasing nocturnal arousals: decreasing the time spent awake following the initial onset of sleep: increasing the total amount of sleep; reducing the fragmentation of sleep: altering the timing, frequency or duration of REM sleep bouts: altering the timing, frequency or duration of slow wave (i.e. stages 3 or 4) sleep bouts, increasing the amount and percentage of stage 2 sleep: promoting slow wave sleep: enhancing EEG-delta activity during sleep: increasing daytime alertness: reducing daytime drowsiness; treating or reducing excessive daytime sleepiness: insomnia: hypersomnia; narcolepsy; interrupted sleep: sleep apnea: wakefulness: nocturnal myoclonus: REM sleep interruptions: jet-lag; shift workers' sleep disturbances: dyssomnias: night terror; insomnias associated with depression: emotional/mood disorders; as well as sleep walking and enuresis; and sleep disorders which accompany aging: Alzheimer's sundowning: conditions associated with circadian rhythmicity as well as mental and physical disorders associated with travel across time zones and with rotating shift-work schedules; conditions due to drugs which cause reductions in REM sleep as a side effect: syndromes which are manifested by non-restorative sleep and muscle pain or sleep apnea which is associated with respiratory disturbances during sleep; and conditions which result from a diminished quality of sleep.

Pain disorders for which the compounds of the invention may be useful include neuropathic pain (such as postherpetic neuralgia, nerve injury, the “dynias”. e.g., vulvodynia, phantom limb pain, root avulsions, painful diabetic neuropathy, painful traumatic mononeuropathy, painful polyneuropathy): central pain syndromes (potentially caused by virtually any lesion at any level of the nervous system): postsurgical pain syndromes (eg, postmastectomy syndrome, postthoracotomy syndrome, stump pain): bone and joint pain (osteoarthritis): repetitive motion pain: dental pain: cancer pain; myofascial pain (muscular injury, fibromyalgia): perioperative pain (general surgery, gynecological): chronic pain; dysmennorhea, as well as pain associated with angina, and inflammatory pain of varied origins (e.g. osteoarthritis, rheumatoid arthritis, rheumatic disease, teno-synovitis and gout); headache; migraine and cluster headache: primary hyperalgesia: secondary hyperalgesia: primary allodynia; secondary allodynia: or other pain caused by central sensitization.

Potential conditions or disorders that have a strong inflammatory component for which the compounds of the invention may be useful include one or more of the following conditions or diseases: diabetes (systemic inflammation in diabetes marked by increases in blood cytokines e g. IL-6 and TNFα which may lead to insulin resistance); asthma: arthritis; cystic fibrosis; sepsis; ulcerative colitis; inflammatory bowel disease; atherosclerosis; neuroinflammation associated with neurodegenerative diseases (e.g. Alzheimer's disease, Parkinson's disease, Creutzfeldt-Jacob disease, frontotemporal dementia, corticobasal degeneration, Pick's disease, progressive supranuclear palsy, traumatic brain injury. Huntington's disease, amyotrophic lateral sclerosis).

Compounds of the invention may also be used to treat or prevent or ameliorate dyskinesia and protect against neurodegeneration in nigrostriatal neurons in Parkinson's disease. Furthermore, compounds of the invention may be used to decrease tolerance and/or dependence to opioid treatment of pain, and for treatment of withdrawal syndrome of e.g., alcohol, opioids, and cocaine.

The compounds of the present invention may be administered in the form of pharmaceutically acceptable salts. The term “pharmaceutically acceptable salt” refers to a salt that possesses the effectiveness of the parent compound and that is not biologically or otherwise undesirable (e.g., is neither toxic nor otherwise deleterious to the recipient thereof). Suitable salts include acid addition salts that may, for example, be formed by mixing a solution of the compound of the present invention with a solution of a pharmaceutically acceptable acid such as hydrochloric acid, sulfuric acid, acetic acid, trifluoroacetic acid, or benzoic acid. Many of the compounds of the invention carry an acidic moiety, in which case suitable pharmaceutically acceptable salts thereof can include alkali metal salts (e.g., sodium or potassium salts), alkaline earth metal salts (e.g., calcium or magnesium salts), and salts formed with suitable organic ligands such as quatemary ammonium salts. Also, in the case of an acid (—COOH) or alcohol group being present, pharmaceutically acceptable esters can be employed to modify the solubility or hydrolysis characteristics of the compound.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties. Selection and Use. (2002) Zurich: Wiley-VCH: S. Berge et al, Journal of Pharmaceutical Sciences (1977) 66 (1): 1-19; P Gould, International J. of Pharmaceutics (1986) 33:201-217; Anderson et al, The Practice of Medicinal Chemistry (1996), Academic Press, New York; and in The Orange Book (Food & Drug Administration, Washington, D.C, on their website).

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

For the purposes of preventing, treating, or ameliorating the cognitive impairments in Alzheimer's disease, Parkinson's disease, schizophrenia, L-DOPA induced-dyskinesia, and inflammation, the compounds of the present invention, optionally in the form of a salt, can be administered by any means that produces contact of the active agent with the agent's site of action. They can be administered by one or more conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered by one or more of the following: orally, parenterally (including subcutaneous injections, intravenous, intramuscular, intraosteal injection or infusion techniques), by inhalation (such as in a spray form), or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as solubility aids. Injectable solutions can be prepared according to methods known in the art wherein the carrier comprises a saline solution, a glucose solution or a solution containing a mixture of saline and glucose. Further description of methods suitable for use in preparing pharmaceutical compositions of the present invention and of ingredients suitable for use in said compositions is provided in Remington's Pharmaceutical Sciences, 18th edition (ed. A. R. Gennaro. Mack Publishing Co., 1990).

The compounds of this invention can be administered orally in a dosage range of 0.001 to 1000 mg/kg of mammal (e.g., human) body weight per day in a single dose or in divided doses. One dosage range is 0.01 to 500 mg/kg body weight per day orally in a single dose or in divided doses. Another dosage range is 0.1 to 100 mg/kg body weight per day orally in single or divided doses. For oral administration, the compositions can be provided in the form of tablets or capsules containing 1.0 to 500 mg of the active ingredient, particularly 1, 5, 10, 15, 20, 25, 50, 75, 100, 150, 200, 250, 300, 400, and 500 mg of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, and the severity of the particular condition.

As noted above, the present invention also relates to a method of preventing, treating, or ameliorating the cognitive impairments in Alzheimer's disease, Parkinson's disease, schizophrenia. L-DOPA induced-dyskinesia, and inflammation with a compound of the present invention in combination with one or more therapeutic agents and a pharmaceutical composition comprising a compound of the present invention and one or more therapeutic agents selected from the group consisting of anti-Alzheimer's Disease agents, for example beta-secretase inhibitors; MI mAChR agonist or PAMs: M4 mAChR agonists or PAMs: mGluR2 antagonists or NAMs or PAMs: ADAM 10 ligands or activators: gamma-secretase inhibitors, such as LY450139 and TAK 070: gamma secretase modulators: tau phosphorylation inhibitors: glycine transport inhibitors: LXR β agonists; ApoE4 conformational modulators: NR2B antagonists; androgen receptor modulators: blockers of Aß oligomer formation: 5-HT4 agonists, such as PRX-03140:5-HT6 antagonists, such as GSK 742467. SGS-518, FK-962, SL-65.0155, SRA-333 and xaliproden: S-HTla antagonists, such as lecozotan, p25/CDKS inhibitors; NKI/NK3 receptor antagonists: COX-2 inhibitors; LRRK2 inhibitors: HMG-COA reductase inhibitors; NSAIDs including ibuprofen: vitamin E; anti-amyloid antibodies (including anti-amyloid humanized monoclonal antibodies), such as bapineuzumab, ACC001. CAD106, AZD3102. HI2AIIVI: anti-inflammatory compounds such as (R)-flurbiprofen, nitroflurbiprofen, ND-1251, VP-025, HT-0712 and EHT-202; PPAR gamma agonists, such as pioglitazone and rosiglitazone: CB-1 receptor antagonists or CB-1 receptor inverse agonists, such as AVE1625; antibiotics such as doxycycline and rifampin: N-methyl-D-aspartate (NMDA) receptor antagonists, such as memantine, neramexane and EVT101: cholinesterase inhibitors such as galantamine, rivastigmine, donepezil, tacrine, phenserine, ladostigil and ABT-089; growth hormone secretagogues such as ibutamoren, ibutamoren mesylate, and capromorelin; histamine H3 receptor antagonists such as ABT-834, ABT 829, GSK 189254 and CEP16795. AMPA agonists or AMPA modulators, such as CX-717, LY 451395, LY404187 and S-18986; PDE IV inhibitors, including MEM1414, HT0712 and AVE8112: GABAA inverse agonists: GSK3B inhibitors, including AZD1080, SAR502250 and CEP16805; neuronal nicotinic agonists; selective MI agonists: HDAC inhibitors; and microtubule affinity regulating kinase (MARK) ligands: or other drugs that affect receptors or enzymes that either increase the efficacy, safety, convenience, or reduce unwanted side effects or toxicity of the compounds of the present invention.

Examples of combinations of the compounds of the instant invention include combinations with agents for the treatment of schizophrenia, for example in combination with sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, aiprazolam, amisulpride, amitriptyline, amobarbital, amoxapine, aripiprazole, bentazepam, benzoctamine, brotizolam, bupropion, busprione, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproelone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, zolpidem, and salts thereof, and combinations thereof, and the like, or the subject compound may be administered in conjunction with the use of physical methods such as with light therapy or electrical stimulation.

In another embodiment, the compounds of the instant invention may be employed in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexol) hydrochloride: COMT inhibitors such as entacapone, MAO-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate.

In another embodiment, the compound of the instant invention may be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothivene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the compounds of the instant invention may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the compounds of the instant invention may be employed in combination with acetophenazine, alentemol, aripiprazole, amisuipride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, Jisuride, loxapine, mesoridazine, molindolone, naxagolide, olanzapine, pergolide, perphenazine, pimozide, pramipexole, quetiapine, risperidone, sulpiride, tetrabenazine, frihexyphenidyl, thioridazine, thiothixene, trifluoperazine or ziprasidone.

Examples of combinations of the compounds of the instant invention include combinations with agents for the treatment of pain, for example non-steroidal anti-inflammatory agents, such as aspirin, diclofenac, duflunisal, fenoprofen, flurbiprofen, ibuprofen, indomethacin, ketoprofen, ketorolac, naproxen, oxaprozin, piroxicam, sulindac and tolmetin; COX-2 inhibitors, such as celecoxib, rofecoxib, valdecoxib, 406381 and 644784: CB-2 agonists, such as 842166 and SAB378: VR-1 antagonists, such as AMG517, 705498, 782443. PAC20030. VI14380 and A425619; bradykinin B1 receptor antagonists, such as SSR240612 and NVPSAA164; sodium channel blockers and antagonists, such as VX409 and SPI860; nitric oxide synthase (NOS) inhibitors (including iNOS and nNOS inhibitors), such as SD6010 and 274150; glycine site antagonists, including lacosamide; neuronal nicotinic agonists, such as ABT 894; NMDA antagonists, such as AZD4282: potassium channel openers: AMPA/kainate receptor antagonists; calcium channel blockers, such as ziconotide and NMED160: GABA-A receptor IO modulators (e.g., a GABA-A receptor agonist); matrix metalloprotease (MMP) inhibitors: thrombolytic agents: opioid analgesics such as codeine, fentanyl, hydromorphone, levorphanol, meperidine, methadone, morphine, oxycodone, oxymorphone, pentazocine, propoxyphene: neutrophil inhibitory factor (NIF): pramipexole, ropinirole: anticholinergics: amantadine; monoamine oxidase BIS (“MAO-B”) inhibitors: 5HT receptor agonists or antagonists: mGlu5 antagonists, such as AZD9272: alpha agonists, such as AGN XX/YY: neuronal nicotinic agonists, such as ABT894: NMDA receptor agonists or antagonists, such as AZD4282: NKI antagonists; selective serotonin reuptake inhibitors (“SSRI”) and/or selective serotonin and norepinephrine reuptake inhibitors (“SSNRI”), such as duloxetine: tricyclic antidepressant drugs, norepinephrine modulators; lithium; valproate; gabapentin: pregabalin; rizatriptan; zolmitriptan; naratriptan and sumatriptan.

The compounds of the present invention may be administered in combination with compounds useful for enhancing sleep quality and preventing and treating sleep disorders and sleep disturbances, including e.g., sedatives, hypnotics, anxiolytics, antipsychotics, antianxiety agents, antihistamines, benzodiazepines, barbiturates, cyclopyrrolones, orexin antagonists, alpha-1 antagonists. GABA agonists, 5HT-2 antagonists including 5HT-2A antagonists and 5HT-2A/2C antagonists, histamine antagonists including histamine H3 antagonists, histamine H3 inverse agonists, imidazopyridines, minor tranquilizers, melatonin agonists and antagonists, melatonergic agents, other orexin antagonists, orexin agonists, prokineticin agonists and antagonists, pyrazolopyrimidines, T-type calcium channel antagonists, triazolopyridines, and the like, such as: adinazolam, allobarbital, alonimid, alprazolam, amitriptyline, amobarbital, amoxapine, armodafinil, APD-125, bentazepam, benzoctamine, brotizolam, bupropion, busprione, butabarbital, butalbital, capromorelin, capuride, carbocloral, chloral betaine, chloral hydrate, chlordiazepoxide, clomipramine, clonazepam, cloperidone, clorazepate, clorethate, clozapine, conazepam, cyprazepam, desipramine, dexclamol, diazepam, dichloralphenazone, divalproex, diphenhydramine, doxepin. EMD-281014, eplivanserin, estazolam, eszopiclone, ethchlorynol, etomidate, fenobam, flunitrazepam, flurazepam, fluvoxamine, fluoxetine, fosazepam, gaboxadol, glutethimide, halazepam, hydroxyzine, ibutamoren, imipramine, indiplon, lithium, lorazepam, lormetazepam. LY-156735, maprotiline, MDL-100907, mecloqualone, melatonin, mephobarbital, meprobamate, methaqualone, methyprylon, midaflur, midazolam, modafinil, nefazodone, NGD-2-73, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, ramelteon, reclazepam, roletamide, secobarbital, sertraline, suproclone, TAK-375, temazepam, thioridazine, tiagabine, tracazolate, tranylcypromaine, trazodone, triazolam, trepipam, tricetamide, triclofos, trifluoperazine, trimetozine, trimipramine, uldazepam, venlafaxine, zaleplon, zolazepam, zopiclone, zolpidem, and salts thereof, and combinations thereof, and the like, or the compound of the present invention may be administered in conjunction with the use of physical methods such as with light therapy or electrical stimulation.

Compounds of the instant invention are useful for the treatment of moderate to severe dementia of the Alzheimer's type alone or in combination with an NMDA receptor antagonist, such as memantine, or in combination with an acetylcholinesterase inhibitor (AChEI) such as donepezil.

Compounds of the instant invention are useful for the treatment of mild to moderate dementia of the Alzheimer's type alone or in combination with either galantamine, rivastigmine, or donepezil.

Compounds of the instant invention are useful for the treatment of dementia associated with Parkinson's disease alone or in combination with rivastigmine.

Compounds of the instant invention are useful for the treatment of motor fluctuations in patients with advanced Parkinson's disease alone or in combination with carbidopa and levodopa

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). A compound of the invention and an additional therapeutic agent may be present in fixed amounts (dosage amounts) in a single dosage unit (e.g., a capsule, a tablet and the like).

The a7 nAChR positive allosteric modulator (PAM) activity of the present compounds may be tested using assays known in the art. The a7 nAChR PAMs described herein have activities in an automated patch-clamp electrophysiology functional assay as described in the examples. The assay was performed using the lonFlux HT in a whole-cell, population patch configuration. See Golden et al. Assay Drug Dev. Technol. (2011) 9:608-619. The compounds were assessed for their ability to modulate the function of the human a7 nAChR stably expressed in a HEK cell line both in the presence, and in the absence of the natural a7 agonist acetylcholine. By performing a series of such measurements at different concentrations, the effective concentration of the a7 nAChR PAMs (EC₅₀) was determined. See Spencer et al. Assay Drug Dev. Technol. (2012) 10:313-324.

The present invention also includes processes for making compounds of formula I. The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above. The following reaction schemes and examples serve only to illustrate the invention and its practice.

### Reaction Schemes

The compounds of the present invention can be prepared readily according to the following schemes and specific examples, or modifications thereof, using readily available starting materials, reagents and conventional synthetic procedures. In these reactions, it is also possible to make use of variants which are themselves known to those of ordinary skill in this art but are not mentioned in greater detail. The general procedures for making the compounds claimed in this invention can be readily understood and appreciated by one skilled in the art from viewing the following schemes.

Key benzylamine intermediates may be prepared according to Scheme 1, in which a Sharpless asymmetric aminohydroxylation of styrene 1.1 is carried out using dichlorohydantoin and potassium osmate (VI)dihydrate in the presence of hydroquinidine 1,4-phthalazinediyl diether. The resulting protected aminoalcohol 1.2 is methylated with Mel and Ag₂O in CH₃CN to afford the corresponding ether 1.3. Alternative reagents, familiar to those skilled in the art of organic synthesis, may be used to methylate 1.2 to 1.3. Compound 1.3 may be deprotected using, for example, HCl in dioxane to afford 1.4 as the hydrochloride salt. Other acidic conditions (for example HCl in Et₂O or TFA) can be employed in the deprotection reaction.

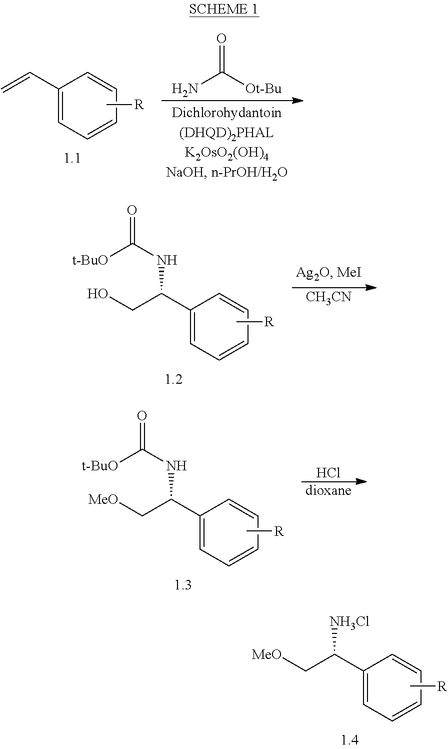

Additional amines of interest may be prepared according to Scheme 2, in which the Ellman sulfinamide chiral auxiliary is used to prepare the amines in enantiomerically enriched form. For example, (R)-(+)-2-methyl-2-propanesulfinamide 2.1 and the protected glycolaldehyde 2.2 may be condensed using PPTS and MgSO₄ in dichloromethane to form sulfinimine 2.3. Other methods of forming the sulfinimine 2.3 may also be employed, such as treating a solution of 2.1 and 2.2 with CuSO₄ or Ti(OEt)₄ in DCM or THF. Reaction of the sulfinimine 2.3 with a lithiated pyridine, obtained via lithium-halogen exchange on bromide 2.4, leads to sulfinamide 2.5. Other organometallic reagents, such as Grignard reagents, may also be employed to give a variety of amine products of interest. Deprotection, for example using HCl, provides the desired amine intermediate 2.6 as the hydrochloride salt. Other acidic conditions (HCl in 1,4-dioxane or TFA, for example) can be employed in the deprotection reaction.

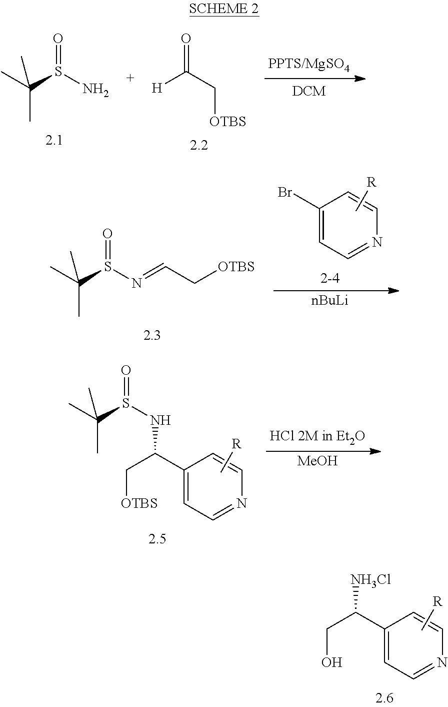

Other useful amine intermediates may be prepared according to Scheme 3 wherein the circle represents a variety of cycloalkyl, heterocyclyl, aryl, or heteroaryl rings. Sulfinimine 3.2 can be obtained by treating aldehyde 3.1 with the Ellman sulfinamide chiral auxiliary in the presence of titanium (IV) ethoxide in tetrahydrofuran. A variety of other reagents and solvents may be used to promote this condensation reaction, including the use of MgSO₄ or CuSO₄ in dichloromethane. Monofluoromethyl and difluoromethyl phenyl sulfone anions, obtained by treatment of 3.3 (X═H or F) with LiHMDS, are reacted with sulfinimine 3.2 to give the corresponding sulfinamides 3.4 with good diastereoselectivity. Reductive removal of the phenyl sulfone moiety can be achieved using sodium-mercury amalgam to afford sulfinamide 3.5 and deprotection is carried out under acidic conditions (for example HCl in 1,4-dioxane) to offer the monofluoromethyl- or difluoromethyl-amine 3.6 (X═H or F).

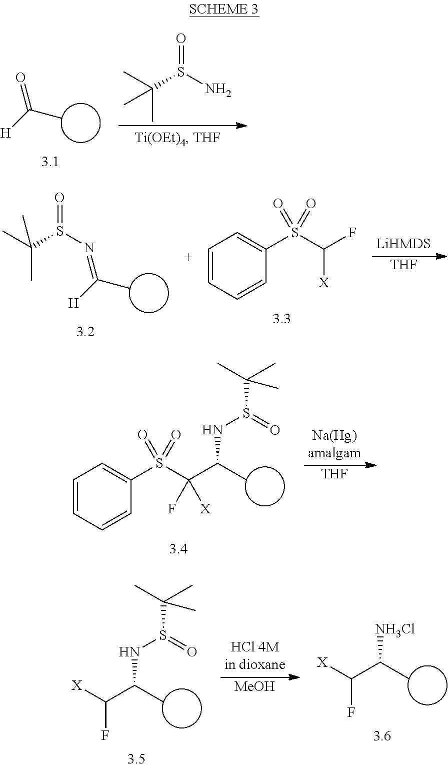

A variety of aryl or heteroaryl cyclopropylamines may be prepared according to Scheme 4, in which the circle represents the aryl or heteroaryl ring. The cyano analogue 4.1 is reacted with ethylmagnesium bromide in the presence of titanium (IV) isopropoxide in tetrahydrofuran to give the corresponding cyclopropylamine 4.2.

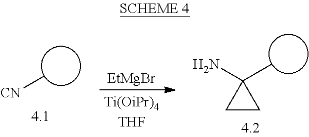

The formation of spirocyclopropyl carboxylic acids that are key synthetic intermediates of the present invention is described in Scheme 5. The ketone 5.1 (X═O or CH₂; Y═CH₂ or a bond) may be employed in a Wittig reaction, in which it is condensed with the ylide derived from methyltriphenylphosphonium bromide and t-BuOK to provide the corresponding alkene 5.2. Cyclopropanation of the resulting alkene 5.2 may be performed using, for example, ethyl diazoacetate in the presence of a suitable catalyst such as copper (I) trifluoromethanesulfonate benzene complex or rhodium (II) acetate dimer to afford the ester 5.3. Standard saponification of 5.3 under basic conditions, for example using lithium hydroxide in aqueous methanol, furnishes the desired carboxylic acid 5.5 (X═O or CH₂; Y═CH₂ or a bond). Other bases can be employed for this hydrolysis step, including sodium or potassium hydroxide. An alternate route to ester 5.3 makes use of Horner-Wadsworth-Emmons methodology, in which ethyl(triphenylphosphoranylidene)acetate is reacted with ketone 5.1 to give ester 5.4, and cyclopropanation of 5.4 using trimethylsulfoxonium iodide and potassium tert-butoxide in dimethylsulfoxide leads to compound 5.3.

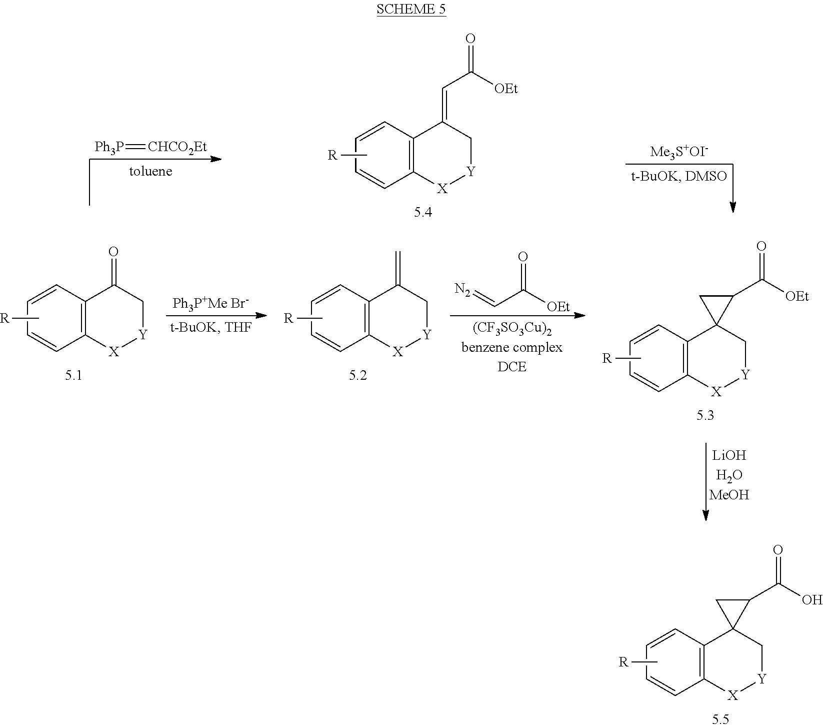

Many compounds of the present invention may be prepared according to Scheme 6, in which acid 5.5 (X═O or CH₂; Y═CH₂ or a bond) is coupled to an amine (WNH₂) using HBTU and Hünig's base in dichloromethane to give the desired amide 6.1. Other coupling conditions, known to those skilled in the art of organic synthesis, including the use of reagents such as DCC, EDC, HATU, HOBt, HOAt and their combinations or T3P, can be employed to provide amide 6.1. Alternatively, the acid 5.5 can be activated by forming the corresponding acid chloride, using oxalyl chloride in the presence of catalytic DMF, or anhydride, using pivaloyl chloride, and the acid chloride or anhydride may be reacted with an amine of interest to afford the corresponding amide. Additionally, amide 6.1 may be obtained by treatment of ester 5.3 with an aluminum amide derived from treating amine WNH₂ with trimethylaluminum. If amide 6.1 is a mixture of enantiomers or diastereomers, the mixture may be separated by chromatography. Alternatively, acid 5.5 and/or amine WNH₂ (for example, amines 1.4, 2.6, 3.6 or 4.2) may be employed as single enantiomers or diastereomers to obtain 6.1 enriched in a single enantiomer or diastereomer.

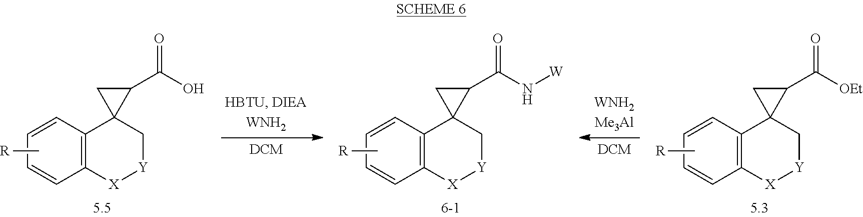

It is understood that the compounds and intermediates in the foregoing reaction schemes may be employed as synthetic intermediates in other schemes that involve similar intermediates to produce alternative compounds of the present invention.

In some cases, the order of carrying out the foregoing reaction schemes may be varied to facilitate the reaction or to avoid unwanted reaction products. Additionally, various protecting group strategies familiar to one skilled in the art of organic synthesis may be employed to facilitate the reaction or to avoid unwanted reaction products.

In some cases, the final product may be further modified, for example, by manipulation of substituents. These manipulations may include, but are not limited to, reduction, oxidation, alkylation, acylation, and hydrolysis reactions which are commonly known to those skilled in the art.

The following examples are provided so that the invention might be more fully understood. These examples are illustrative only and should not be construed as limiting the invention in any way. Wherein a racemic mixture is produced, the enantiomers may be separated using SFC reverse or normal phase chiral resolution conditions either after isolation of the final product or at a suitable Intermediate, followed by processing of the single isomers individually. It is understood that alternative methodologies may also be employed in the synthesis of these key intermediates and examples. Asymmetric methodologies (e.g. chiral catalysis, auxiliaries) may be used where possible and appropriate. The exact choice of reagents, solvents, temperatures, and other reaction conditions, depends upon the nature of the intended product. Unless otherwise indicated, when ratios of compounds (such as for examples solvents) are given, the ratio is on a volume to volume basis. For example, a 20:80 mixture of ethyl acetate:petroleum ether means a mixture of 20 parts by volume ethyl acetate to 80 parts by volume of petroleum ether. Additionally, unless otherwise specifically indicated, all reagents are commercially available, known in the literature, or readily synthesized by one skilled in the art. Straightforward protecting group strategies were applied in some routes.

The following abbreviations are used throughout the text:
Ac acetyl
app apparent
aq aqueous
Ar aryl
BINAP 2,2′-bis(diphenylphosphino)- 1,1′-binaphthalene
Bn benzyl
Boc tert-butoxycarbonyl
br broad
Bu butyl
ca circa (approximately)
Cbz carboxybenzyl
CDI 1,1′-carbonyldiimidazole
d doublet
DCC
DCDMH 1,3-dichloro-5,5-dimethylhydantoin
DCE 1,2-dichloroethane
DCM dichloromethane
dd doublet of doublets
(DHQD)₂PHAL hydroquinidine 1,4-phthalazinediyl diether
DIBAL diisobutylaluminum hydride
DIAD diisopropyl azodicarboxylate
DIEA N,N-diisopropylethylamine
DMAP 4-(dimethylamino)pyridine
DMF N,N-dimethylformamide
DMF-DMA N,N-dimethylformamide dimethylacetal
DMSO dimethylsulfoxide
EDC N-(3-dimethylaminopropyl)-N′-ethylcarbodiimide hydrochloride
eq equivalents
ESI electrospray ionization
Et ethyl
h hours
HATU O-(7-azabenzotriazol -1-yl)-N,N,N′N′-tetramethyluronium
HBTU O-(benzotrialzon-1-yl)-N,N,N′N′-tetramethyluronium hexafluorophosphate
HMDS hexamethyldisilazane
HOAt 1-hydroxy-7-azabenzotriazole
HOBt 1-hydroxybenzotriazole
HPLC high performance liquid chromatography
Hz hertz
i-Pr isopropyl
J coupling constant
LAH lithium aluminum hydride
LCMS liquid chromatography-mass spectrometry
LDA lithium diisopropylamide
m/z mass to charge ratio
m multiplet
mCPBA 3-chloroperoxybenzoic acid
Me methyl
min minutes
Ms methanesulfonyl
MW molecular weight
n-BuLi n-butyllithium
n-HexLi n-hexyllithium
MR nuclear magnetic resonance
OAc acetate
p pentet
Pd/C palladium on carbon
Ph phenyl
PPTS pyridinium 4-toluenasulfonate
Pr propyl
psi pounds per square inch
p-Ts 4-toluenesulfonyl
PTSA 4-toluensulfonic acid
Py pyridyl
q quartet
RockPhos Pd G3 [(2-Di-tert-butylphosphino-3-methoxy-6-methyl-2′,4′,6′-triisopropyl-1,1′-biphenyl)-2-(2-aminobiphenyl)]palladium(II) methanesulfonate
rt ambient temperatures
s singlet
SM starting material
SPE solid phase extraction
t triplet
td triplet of doublets
TBS tert-butyldimethylsilyl
t-Bu tert-butyl
TCCA trichloroisocyanuric acid
TEA triethylamine
TFA trifluoroacetic acid
Tf trifluoromethanesulfonyl
THF tetrahydrofuran
T3P 2,4,6-tripropyl-1,3,5,2,4,6-trioxatriphosphorinane-2,4,6-trioxide
V/V volume to volume
X-Phos 2-dicyclohexylphosphino-2′,4′,6′-triisopyropylbiphenyl
Xantphos (9,9-dimethyl-9H-xanthene -4,5-diyl)bis(diphenylphosphane)

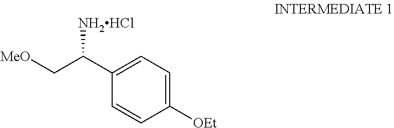

### (1R)-1-(4-Ethoxyphenyl)-2-Methoxyethanamine Hydrochloride

### Step A: Tert-Butyl[(1R)-1-(4-Ethoxyphenyl)-2-Hydroxyethyl]Carbamate

To a stirred solution of tert-butyl carbamate (12.17 g, 103.9 mmol) in n-propanol (100 mL) at 0° C. was added aqueous sodium hydroxide (0.5 M, 150 mL, 75.0 mmol) dropwise. The resulting mixture was allowed to stir at 0° C.′ for 10 min and then dichlorohydantoin (10.17 g, 51.62 mmol) was added portionwise. The resulting mixture was allowed to stir at 0° C. for 10 min and then a solution of hydroquinidine 1,4-phthalazinediyl diether (790 mg, 1.01 mmol) in n-propanol (100 mL) was added dropwise. The resulting mixture was allowed to stir at 0° C. for 15 min and then 4-ethoxystyrene (5.0 g, 33.74 mmol) was added, followed by a solution of potassium osmate (VI)dihydrate (250 mg, 0.67 mmol) in aqueous sodium hydroxide (0.5 M, 50 mL, 25.0 mmol). The reaction mixture was stirred at 0° C. for 6 h and then diluted with water (300 mL). The resulting mixture was extracted with diethyl ether (2×200 mL) and the combined organic extracts were washed with a saturated aqueous solution of sodium chloride (100 mL), dried (magnesium sulfate), filtered, and concentrated under reduced pressure. The residue was purified by silica gel chromatography, eluting with a gradient of ethyl acetate: hexanes ranging from 0:100 to 100:0 to afford the title compound. MS: miz=304.3 [M+Na].

### Step B: Tert-Butyl[(1R)-1-(4-Ethoxyphenyl)-2-Methoxyethyl]Carbamate

To a stirred solution of tert-butyl [(1R)-1-(4-ethoxyphenyl)-2-hydroxyethyl]carbamate (2.10 g, 7.46 mmol) in acetonitrile (30 mL) at 0° C. was added silver (I) oxide (8.65 g, 37.32 mmol) portionwise followed by iodomethane (2.32 mL, 37.32 mmol) dropwise. The reaction mixture was allowed to warm to ambient temperature and stirred for 60 h. The reaction mixture was filtered through a pad of CeliteR and concentrated under reduced pressure. The residue was purified by silica gel chromatography, eluting with a gradient of ethyl acetate: hexanes ranging from 0:100 to 50:50 to afford the title compound. MS: m′z=318.3 [M+Na].

### Step C: (1R)-1-(4-Ethoxyphenyl)-2-Methoxyethanamine Hydrochloride

To a stirred solution of tert-butyl [(1R)-1-(4-ethoxyphenyl)-2-methoxyethyl]carbamate (2.76 g, 9.35 mmol) in 1,4-dioxane (20 mL) at ambient temperature was added a solution of hydrochloric acid in 1,4-dioxane (4.0 M, 34 mL, 136 mmol) dropwise. The reaction mixture was stirred for 4 h and then concentrated under reduced pressure. The residue was crystallized from diethyl ether to afford the title compound. MS: m==179.2 [M-NH₂].

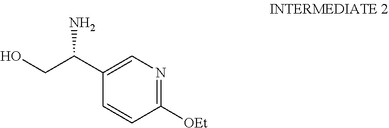 [SMILES: CCOc1ccc([C@@H](N)CO)cn1]

### (2R)-2-Amino-2-(6-Ethoxypyridin-3-Yl) Ethanol

### Step A: (S)-N-[2-{[Tert-Butyl(Dimethyl) Silyl]Oxy}Ethylidene]-2-Methylpropane-2-Sulfinamide

To a stirred solution of (R)-(+)-2-methyl-2-propanesulfinamide (2.3 g. 19.12 mmol) in dichloromethane (5 mL) at ambient temperature were added magnesium sulfate (11.5 g. 95.6 mmol) and pyridinium p-toluenesulfonate (240 mg, 0.95 mmol) followed by a solution of (tert-butyldimethylsilyloxy) acetaldehyde (5.0 g. 28.68 mmol) in dichloromethane (2 mL). The reaction mixture was allowed to stir at ambient temperature for 24 h. The mixture then was diluted with ethyl acetate (20 mL), filtered through a pad of Celite™, and concentrated under reduced pressure. The residue was purified by silica gel chromatography, eluting with a gradient of ethyl acetate: hexanes ranging from 0:100 to 10:90 to afford the title compound. ¹H NMR (300 MHz, CDCl₃): δ 8.05 (t, J=3.1 Hz, 1H): 4.54 (d, J=3.1 Hz, 2H); 1.20 (s, 9H): 0.91 (s. 9 H); 0.09 (s, 6H).

### Step B: (S)-N-[(1R)-2-{[Tert-Butyl(Dimethyl) Silyl]Oxy}-1-(6-Ethoxypyridin-3-Vl)Ethyl]-2-Methylpropane-2-Sulfinamide

To a stirred solution of 5-bromo-2-ethoxypyridine (2.30 g, 11.3 mmol) in tetrahydrofuran (20 mL) at −78° C. was added a solution of n-butyllithium (2.0 M in cyclohexane, 12 mL, 23.7 mmol) dropwise. The resulting mixture was stirred at −78° C. for 1 h and a solution of(S)-N-[2-{[tert-butyl(dimethyl) silyl]oxy}ethylidene]-2-methylpropane-2-sulfinamide (3.3 g, 11.3 mmol) in tetrahydrofuran (20 mL) was added dropwise at −78° C. The reaction mixture was allowed to warm to ambient temperature and allowed to stir for 20 h. The resulting mixture was diluted with a saturated aqueous solution of ammonium chloride (50 mL) and extracted with ethyl acetate (3×50 mL). The combined organic extracts were washed with a saturated aqueous solution of sodium chloride (25 mL), dried (magnesium sulfate), filtered, and concentrated under reduced pressure. The residue was purified by silica gel chromatography, eluting with a gradient of ethyl acetate: hexanes ranging from 0:100 to 30:70 to afford the title compound. JH NMR (300 MHz, CDCl₃): δ 8.10 (d, J=2.4 Hz, 1H); 7.50 (dd, J=8.6 Hz, 2.4 Hz, 1H); 6.69 (d, J=8.6 Hz, 1H): 4.47 (m, 1H): 4.34 (q, J=7.1 Hz, 2H): 4.24 (bs, 1H): 3.75 (m. 1 H): 3.58 (m. 1 H); 1.38 (t. J=7.1 Hz. 3 H); 1.21 (s, 9H); 0.89 (s, 9H); 0.04 (s. 6 H).

### Step C: (2R)-2-Amino-2-(6-Ethoxypyridin-3-Yl) Ethanol

To a stirred solution of(S)-N-[(1R)-2-{[tert-butyl(dimethyl) silyl]oxy}-1-(6-ethoxypyridin-3-yl)ethyl]-2-methylpropane-2-sulfinamide (2.0 g, 5.0 mmol) in methanol (20 mL) at ambient temperature was added a solution of hydrochloric acid in diethyl ether (2.0 M. 25 mL, 50.0 mmol) dropwise and the reaction mixture was stirred at ambient temperature for 18 h. The resulting mixture was adjusted to approximately pH 8 using a solution of ammonia in methanol (7.0 M) and was concentrated under reduced pressure. The residue was purified by silica gel chromatography, eluting with a gradient of ammonia in methanol (7.0 M): ethyl acetate ranging from 0:100 to 20:80, to afford the title compound. MS: m′z=183.1 [M+H].

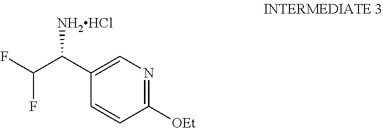

### (1R)-1-(6-Ethoxypyridin-3-Yl)-2,2-Difluoroethanamine Hydrochloride

### Step A: (S)-N-[(6-Ethoxypyridin-3-yl)Methylidene]-2-Methylpropane-2-Sulfinamide

To a stirred solution of 6-ethoxynicotinaldehyde (480 mg, 3.17 mmol) and(S)-(−)-2-methyl-2-propanesulfinamide (385 mg, 3.17 mmol) in tetrahydrofuran (10 mL) at ambient temperature was added titanium (IV) ethoxide (1.4 mL. 6.35 mmol) dropwise. The reaction mixture was allowed to stir at ambient temperature for 24 h and was then diluted with a saturated aqueous solution of sodium chloride (20 mL). The resulting mixture was filtered through a pad of Celite® and the filtrate was extracted with ethyl acetate (3×50 mL). The combined organic extracts were washed with a saturated aqueous solution of sodium chloride (20 mL), dried (magnesium sulfate), filtered, and concentrated under reduced pressure to afford the title compound in sufficient purity for use in the next step. ¹H NMR (300 MHz, CDCl₃): δ 8.55 (s. 1 H); 8.51 (d, J=2.4 Hz, 1H): 8.12 (dd, J=8.6 Hz, 2.4 Hz, 1H): 6.81 (d, J=8.6 Hz, 1H); 4.43 (q, J=7.1 Hz, 2H): 1.42 (t, J=7.1 Hz, 3H): 1.24 (s, 9H).

### Step B: (S)-N-[(1R)-1-(6-Ethoxypyridin-3-Yl)-2,2-Difluoro-2-(Phenylsulfonyl) Ethvl]-2-Methylpropane-2-Sulfinamide

To a stirred solution of(S)-N-[(6-ethoxypyridin-3-yl)methylidene]-2-methylpropane-2-sulfinamide (700 mg. 2.75 mmol) and difluoromethyl phenyl sulfone (530 mg, 2.75 mmol) in tetrahydrofuran (15 mL) at −78° C. was added a solution of lithium bis(trimethylsilyl)amide in tetrahydrofuran (1.0 M, 3.0 mL, 3.0 mmol) dropwise. The reaction mixture was stirred at −78° C. for 2 h. The mixture was diluted with a saturated aqueous solution of ammonium chloride (10 mL) and water (10 mL) and extracted with ethyl acetate (3×25 mL). The combined organic extracts were washed with a saturated aqueous solution of sodium chloride (10 mL), dried (magnesium sulfate), filtered, and concentrated under reduced pressure. The residue was purified by silica gel chromatography, eluting with a gradient of ethyl acetate: hexanes ranging from 0:100 to 50:50 to afford the title compound. MS: m z=447.3 [M+H].

### Step C: (S)-N-[(1R)-1-(6-Ethoxypyridin-3-Yl)-2,2-Difluoroethyl]-2-Methylpropane-2-Sulfinamide

To a stirred solution of(S)-N-[(1R)-1-(6-ethoxypyridin-3-yl)-2,2-difluoro-2-(phenylsulfonyl)ethyl]-2-methylpropane-2-sulfinamide (1.08 g. 2.41 mmol) in methanol (30 mL) at −20° C. was added disodium hydrogen phosphate (2.74 g, 19.3 mmol) followed by sodium mercury amalgam (Na: 20%, 2.2 g, 15.3 mmol). The reaction mixture was stirred for 90 min at −20° C., filtered through a pad of Celite™ and the filtrate was concentrated under reduced pressure. The residue was diluted with water (10 mL) and extracted with ethyl acetate (3×25 mL). The combined organic extracts were washed with a saturated aqueous solution of sodium chloride (10 mL), dried (magnesium sulfate), filtered, and concentrated under reduced pressure to afford the title compound in sufficient purity for use in the next step. ¹H NMR (300 MHZ, CDCl₃): δ 8.16 (d, J=2.4 Hz, 1H): 7.62 (dd, J=8.6 Hz. 2.4 Hz, 1H): 6.76 (d, J=8.6 Hz, 1H); 5.97 (td, J=55.5 Hz. 3.1 Hz, 1H); 4.61 (m, 1H); 4.36 (q. J=7.1 Hz. 2 H); 1.39 (t. J=7.1 Hz. 3 H); 1.23 (s. 9 H).

### Step D: (1R)-1-(6-Ethoxypyridin-3-Vl)-2,2-Difluoroethanamine Hydrochloride

To a stirred solution of(S)-N-[(1R)-1-(6-ethoxypyridin-3-yl)-2,2-difluoroethyl]-2-methylpropane-2-sulfinamide (620 mg. 2.0 mmol) in methanol (15 mL) at ambient temperature was added a solution of hydrochloric acid in 1,4-dioxane (4.0 M, 2.5 mL, 10.0 mmol) dropwise. The reaction mixture was stirred at ambient temperature for 6 h then concentrated under reduced pressure. The residue was crystallized from diethyl ether to afford the title compound. MS: mz=203.2 [M+H]

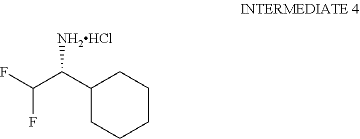

### (1R)-1-Cyclohexyl-2,2-Difluoroethanamine Hydrochloride

Following analogous procedures to those described in Intermediate 3, but using cyclohexanecarboxaldehyde in place of 6-ethoxynicotinaldehyde, the title compound was obtained. ¹H NMR (300 MHz, DMSO-d₆): δ 8.58 (bs, 3H): 6.40 (td, J=53.3 Hz, 2.5 Hz, 1H); 3.40 (m, 1H); 1.80-1.50 (m, 6H); 1.26-1.03 (m, 5H).

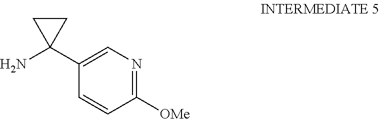 [SMILES: COc1ccc(C2(N)CC2)cn1]

### 1-(6-Methoxypyridin-3-Yl) Cyclopropanamine

To a stirred solution of titanium (IV) ethoxide (1.65 mL, 5.55 mmol) in tetrahydrofuran (20 mL) at 50° C. were added 2-methoxy-5-cyanopyridine (500 mg, 3.7 mmol) and then a solution of ethylmagnesium bromide in tetrahydrofuran (1.0 M, 9.3 mL, 9.3 mmol) dropwise. The reaction mixture was allowed to stir at 50° C. for 3 h. After cooling to ambient temperature, the mixture was acidified with an aqueous solution of hydrochloric acid (3 N. 10 mL, 30 mmol), washed with ethyl acetate (3×10 mL), and these organic extracts were discarded. The aqueous phase was then adjusted to approximately pH 10 by addition of an aqueous solution of sodium hydroxide (1 N) and extracted with ethyl acetate (3×10 mL). These combined organic extracts were washed with a saturated aqueous solution of sodium chloride (20 mL), dried (magnesium sulfate), filtered, and concentrated under reduced pressure. The residue was purified by silica gel chromatography, eluting with a gradient of ethyl acetate: hexanes ranging from about 0:100 to 100:0 to afford the title compound. MS: m/z=165.1 [M+H].

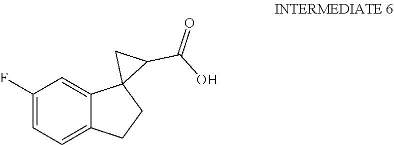 [SMILES: O=C(O)C1CC12CCc1ccc(F)cc12]

### 2′,3′-Dihydrospiro[Cyclopropane-1,1′-Indene]-2-Carboxylic Acid

### Step A: 1-Methylene-2,3-Dihydro-1H-Indene

To a stirred solution of 1-indanone (2.07 g, 15.7 mmol) in tetrahydrofuran (50 mL) at ambient temperature was added triphenylmethylphosphonium bromide (6.72 g, 18.8 mmol). To the resulting stirred suspension was added a solution of potassium fert-butoxide (2.11 g. 18.8 mmol) in tetrahydrofuran (50 mL) dropwise over 30 min. The reaction mixture was stirred at ambient temperature for 18 h and concentrated under reduced pressure. The residue was purified by silica gel chromatography, eluting with a gradient of ethyl acetate: hexanes ranging from 0:100 to 10:90 to afford the title compound. ¹H NMR (300 MHZ, CDCl₃): $7.49 (m. 1 H): 7.35-7.18 (m. 3 H); 5.45 (t. J=2.5 Hz, 1H): 5.03 (t. J=2.1 Hz, 1H): 2.98 (m, 2H); 2.80 (m. 2 H).

### Step B: Ethyl 2′,3′-Dihydrospiro[Cyclopropane-1,1′-Indene]-2-Carboxylate

To a stirred solution of 1-methylene-2,3-dihydro-1H-indene (1.36 g, 10.4 mmol) and rhodium (II) acetate dimer (19 mg, 0.04 mmol) in dichloromethane (50 mL) at 50° C. was added ethyldiazoacetate (1.2 mL, 11.4 mmol) dropwise. The reaction mixture was stirred at 50° C. for 1 h and then at ambient temperature for 2 h. The solution was concentrated under reduced pressure and the residue was purified by silica gel chromatography, eluting with a gradient of ethyl acetate: hexanes ranging from 0:100 to 10:90 to afford the title compound as a mixture of diastereomers. MS: m/z=217.0 [M+H].

### Step C: 2′ 3′-Dihydrospiro[Cyclopropane-1,1′-Indene]-2-Carboxylic Acid

To a stirred solution of ethyl 2′,3′-dihydrospiro[cyclopropane-1, 1′-indene]-2-carboxylate (1.55 g, 7.16 mmol) in ethanol (50 mL) and water (10 mL) at ambient temperature was added lithium hydroxide monohydrate (1.47 g, 35.8 mmol). The reaction mixture was stirred at ambient temperature for 18 h. The solution was concentrated under reduced pressure and the residue was diluted with water (30 mL), adjusted to pH<2 by addition of aqueous hydrochloric acid (6 N), and extracted with diethyl ether (3×50 mL). The combined organic extracts were washed with a saturated aqueous solution of sodium chloride (30 mL), dried (magnesium sulfate), filtered, and concentrated under reduced pressure to afford the title compound as a mixture of diastereomers in sufficient purity for use in the next step. MS: m/z=186.9 [M−H].

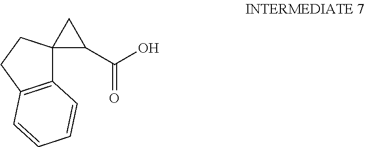 [SMILES: O=C(O)C1CC12CCc1ccccc12]

### Cis-2′,3′-Dihydrospiro[Cyclopropane-1,1′-Indene]-2-Carboxylic Acid

The mixture of diastereomers of 2′,3′-dihydrospiro[cyclopropane-1,1′-indene]-2-carboxylic acid (Intermediate 6) was separated by semi-preparative reversed-phase HPLC, eluting with acetonitrile: water: formic acid (30:70:0.1). The first major peak to elute was cis-2′,3′-dihydrospiro[cyclopropane-1,1′-indene]-2-carboxylic acid, the title compound, and the second major peak to elute was trans-2′,3′-dihydrospiro[cyclopropane-1,1′-indene]-2-carboxylic acid. MS: m/z=186.9 [M−H].

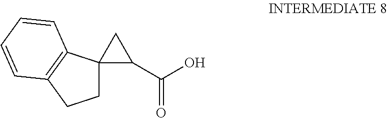 [SMILES: O=C(O)C1CC12CCc1ccccc12]

### Trans-2′,3′-Dihydrospiro[Cyclopropane-1,1′-Indene]-2-Carboxylic Acid

The mixture of diastereomers of 2′,3′-dihydrospiro[cyclopropane-1,1′-indene]-2-carboxylic acid (Intermediate 6) was separated by semi-preparative reversed-phase HPLC, eluting with acetonitrile: water: formic acid (30:70:0.1). The first major peak to elute was cis-2′,3′-dihydrospiro[cyclopropane-1, 1′-indene]-2-carboxylic acid and the second major peak to elute was trans-2′,3′-dihydrospiro[cyclopropane-1,1′-indene]-2-carboxylic acid, the title compound. MS: m/z=186.9 [M−H].

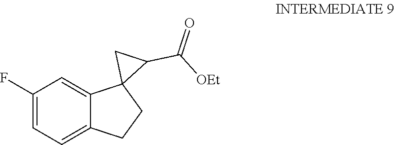 [SMILES: CCOC(=O)C1CC12CCc1ccc(F)cc12]

### Ethyl 6′-Fluoro-2′,3′-Dihydrospiro[Cyclopropane-1,1′-Indene]-2-Carboxylate

### Step A: 6-Fluoro-1-Methylidene-2,3-Dihydro-1H-Indene

Following analogous procedures to those described in Intermediate 6, Step A, but using 6-fluoro-1-indanone in place of 1-indanone, the title compound was obtained. MS: miz=147.21 [M−H].

### Step B: Ethyl 6-Fluoro-2′ 3′-Dihydrospiro[Cyclopropane-1,1′-Indene]-2-Carboxylate

To a solution of 6-fluoro-1-methylene-2,3-dihydro-1H-indene (15.0 g, 101 mmol) in 1,2-dichloroethane (200 mL) was added copper (I) trifluoromethanesulfonate benzene complex (1.0 g, 2.0 mmol) and the mixture was stirred at ambient temperature for 30 min. The resulting mixture was cooled to 0° C. and ethyl diazoacetate (23 mL, 202 mmol) was added dropwise over a period of 1 h. The reaction mixture was stirred at ambient temperature for 18 h then diluted with water (100 mL). The resulting mixture was extracted with dichloromethane (2×200 mL). The combined organic extracts were washed with a saturated aqueous solution of sodium chloride (100 mL), dried (magnesium sulfate), filtered, and concentrated under reduced pressure. The residue was purified by silica gel chromatography, eluting with ethyl acetate:petroleum ether (10:90) to afford the title compound as a mixture of diastereomers. GC-MS: m/z=234.1 [M].

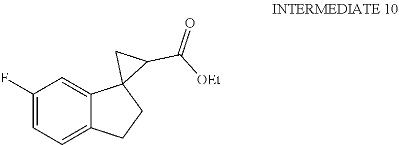 [SMILES: CCOC(=O)C1CC12CCc1ccc(F)cc12]

### Ethyl 6′-Fluoro-2′,3′-Dihydrospiro[Cyclopropane-1,1′-Indene]-2-Carboxylate, Mixture a

The mixture of diastereomers of ethyl 6′-fluoro-2′,3′-dihydrospiro[cyclopropane-1, 1′-indene]-2-carboxylate (Intermediate 9) was separated by SFC, utilizing a Lux A1 column and eluting with methanol: carbon dioxide (20:80). The first major peak to elute was ethyl 6′-fluoro-2′,3′-dihydrospiro[cyclopropane-1, 1′-indene]-2-carboxylate, mixture A, the title compound, and the second major peak to elute was ethyl 6′-fluoro-2′,3′-dihydrospiro[cyclopropane-1, 1′-indene]-2-carboxylate, mixture B. ¹H NMR (300 MHZ, DMSO-de); δ 7.20 (m, 1H); 6.95 (td, J=6.6 Hz, 1.8 Hz, 1H): 6.85 (dd, J=7.5 Hz, 1.8 Hz, 1H): 4.00-3.86 (m. 2 H); 2.95-2.81 (m, 2H): 2.36-2.20 (m, 2H); 2.05-1.98 (m. 1 H); 1.73 (m, 1H); 1.49 (m, 1H); 1.05 (t, J=7.0 Hz, 3H).

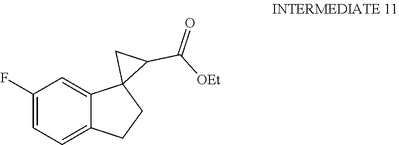 [SMILES: CCOC(=O)C1CC12CCc1ccc(F)cc12]

### Ethyl 6′-Fluoro-2′,3′-Dihydrospiro[Cyclopropane-1,1′-Indene]-2-Carboxylate, Mixture B

The mixture of diastereomers of ethyl 6′-fluoro-2′,3′-dihydrospiro[cyclopropane-1, 1′-indene]-2-carboxylate (Intermediate 9) was separated by SFC, utilizing a Lux A1 column and eluting with methanol: carbon dioxide-20:80. The first major peak to elute was ethyl 6′-fluoro-2′,3′-dihydrospiro[cyclopropane-1, 1′-indene]-2-carboxylate, mixture A, and the second major peak to elute was ethyl 6-fluoro-2′,3′-dihydrospiro[cyclopropane-1,1′-indene]-2-carboxylate, mixture B, the title compound. ¹H NMR (300 MHZ, DMSO-d₆): δ 7.21 (dd, J=6.0 Hz, 4.2 Hz, 1H): 6.95 (td, J=7.0 Hz, 1.8 Hz, 1H); 6.79 (dd, J=7.2 Hz, 1.8 Hz, 1H); 4.10 (q, J=5.1 Hz, 2H); 2.97-2.86 (m. 2 H); 2.17 (t, J=5.7 Hz, 2H): 2,11 (t, J=5.4 Hz, 1H): 1.49 (d, J=5.4 Hz, 2H): 1.05 (t, J=5.4 Hz, 3H).

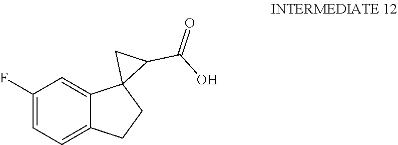 [SMILES: O=C(O)C1CC12CCc1ccc(F)cc12]

### 6′-Fluoro-2′,3′-Dihydrospiro[Cyclopropane-1,1′-Indene]-2-Carboxylic Acid, Mixture a

To a stirred solution of ethyl 6′-fluoro-2′,3′-dihydrospiro[cyclopropane-1,1′-indene]-2-carboxylate, mixture A, (Intermediate 10) (6.0 g, 25.6 mmol) in methanol (50 mL) and water (50 mL), was added LiOH (3.20 g, 76.9 mmol) and the reaction mixture was stirred at ambient temperature for 14 h. Most of the methanol was removed under reduced pressure and the residual mixture was adjusted to approximately pH 4 by addition of aqueous hydrochloric acid (1.5 N), and extracted with ethyl acetate (3×300 mL). The combined organic extracts were washed with water, then a saturated aqueous solution of sodium chloride (30 mL), dried (sodium sulfate), filtered, and concentrated under reduced pressure. The residue was purified by silica gel chromatography, eluting with dichloromethane:methanol (90:10) to afford the title compound. MS: m/z=205.1 [M−H].

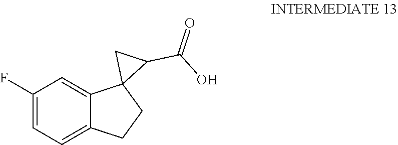 [SMILES: O=C(O)C1CC12CCc1ccc(F)cc12]

### 6′-Fluoro-2′,3′-Dihydrospiro[Cyclopropane-1,1′-Indene]-2-Carboxylic Acid, Diastereomer a

The racemic mixture of 6′-fluoro-2′,3′-dihydrospiro[cyclopropane-1,1′-indene]-2-carboxylic acid, mixture A. (Intermediate 12) was further purified by SFC, utilizing a Lux® A1 column (Phenomenex Inc., Torrance, CA, USA) and eluting with methanol: carbon dioxide (20:80). The first major peak to elute was ethyl 6′-fluoro-2′,3′-dihydrospiro[cyclopropane-1,1′-indene]-2-carboxylate, diastereomer A, the title compound, and the second major peak to elute was ethyl 6′-fluoro-2′,3′-dihydrospiro[cyclopropane-1, 1′-indene]-2-carboxylate, diastereomer B. MS: m/z=205.1 [M−H].

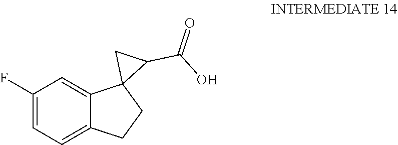 [SMILES: O=C(O)C1CC12CCc1ccc(F)cc12]

### 6′-Fluoro-2′,3′-Dihydrospiro[Cyclopropane-1,1′-Indene]-2-Carboxylic Acid, Diastereomer B

The racemic mixture of 6′-fluoro-2′,3′-dihydrospiro[cyclopropane-1,1′-indene]-2-carboxylic acid, mixture A, (Intermediate 12) was further purified by SFC, utilizing a Lux® A1 column and eluting with methanol: carbon dioxide (20:80). The first major peak to elute was ethyl 6′-fluoro-2′,3′-dihydrospiro[cyclopropane-1,1′-indene]-2-carboxylate, diastereomer A, and the second major peak to elute was ethyl 6′-fluoro-2′,3′-dihydrospiro[cyclopropane-1,1′-indene]-2-carboxylate, diastereomer B, the title compound. MS: m/z=205.1 [M−H].

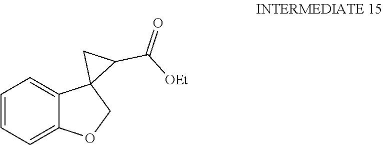 [SMILES: CCOC(=O)C1CC12COc1ccccc12]

### Ethyl 2H-Spiro[1-Benzofuran-3,1′-Cyclopropane]-2′-Carboxylate

Following analogous procedures to those described in Intermediate 9, but using benzofuran-3 (2H)-one in place of 6-fluoro-1-indanone, the title compound was obtained. ¹H NMR (300 MHz, DMSO-d₆): δ 7.15-7.08 (m, 2H); 6.83-6.77 (m, 2H); 4.56-4.42 (m, 2H); 4.06-3.92 (m, 2H); 2.33 (t, J=7.5 Hz, 1H); 1.78-1.73 (m, 1H); 1.62-1.55 (m, 1H); 1.08 (t, J=7.0 Hz, 3H).

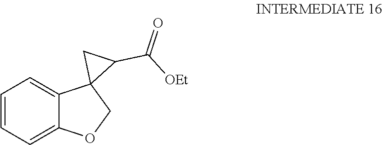 [SMILES: CCOC(=O)C1CC12COc1ccccc12]

### Ethyl 2H-Spiro[1-Benzofuran-3,1′-Cyclopropane]-2′-Carboxylate, Mixture a

The mixture of diastereomers of ethyl 2H-spiro[1-benzofuran-3,1′-cyclopropane]-2′-carboxylate (Intermediate 15) was separated by preparative reversed-phase HPLC, eluting with a gradient of acetonitrile: water: formic acid ranging from 5:95:0.1 to 95:5:0.1. The first major peak to elute was ethyl 2H-spiro[1-benzofuran-3,1′-cyclopropane]-2′-carboxylate, mixture A, the title compound, and the second major peak to elute was ethyl 2H-spiro[1-benzofuran-3,1′-cyclopropane]-2′-carboxylate, mixture B. ¹H NMR (300 MHZ, DMSO-d₆): δ 7.14-7.08 (m, 2 H); 6.82-6.78 (m, 2H); 4.52 (d, J=9.3 Hz, 1H): 4.44 (d. J=9.3 Hz, 1 H); 4.05-3.93 (m, 2 H); 2.34 (t. J=6.6 Hz, 1H): 1.77-1.73 (m, 1H): 1.61-1.58 (m, 1H): 1.06 (t, J=7.0 Hz, 3 H).

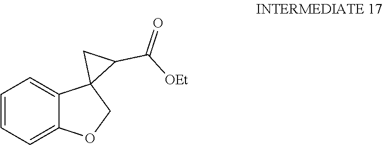 [SMILES: CCOC(=O)C1CC12COc1ccccc12]

### Ethyl 2H-Spiro[1-Benzofuran-3,1′-Cyclopropane]-2′-Carboxylate, Mixture B

The mixture of diastereomers of ethyl 2H-spiro[1-benzofuran-3,1′-cyclopropane]-2′-carboxylate (Intermediate 15) was separated by preparative reversed-phase HPLC, eluting with a gradient of acetonitrile: water: trifluoroacetic acid ranging from 5:95:0.1 to 95:5:0.1. The first major peak to elute was ethyl 2H-spiro[1-benzofuran-3,1′-cyclopropane]-2′-carboxylate, mixture A, and the second major peak to elute was ethyl 2H-spiro[1-benzofuran-3,1′-cyclopropane]-2′-carboxylate, mixture B, the title compound. ¹H NMR (400 MHZ, DMSO-d₆): δ 7.15-7.11 (m, 1H): 6.98-6.96 (m, 1H): 6.87-6.79 (m, 2H): 4.57 (d. J=9.9 Hz, 1H); 4.44 (d, J=9.9 Hz, 1 H); 4.14 (q, J=7.1 Hz, 2H); 2.27-2.24 (m, 1H); 1.68-1.64 (m, 1H); 1.55-1.52 (m, 1H); 1.21 (t, J=7.1 Hz, 3H).

The intermediates appearing in TABLE INT-A were prepared by analogy to the above intermediates, as described or prepared as a result of similar transformations with modifications known to those skilled in the art. The requisite starting materials were described herein, commercially available, known in the literature, or readily synthesized by one skilled in the art. Straightforward protecting group strategies were applied in some routes.

TABLE INT-A

| Intermediate | Structure | Stereochemistry | MS [M + H] |
|---|---|---|---|
| A1 | 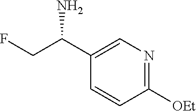 [SMILES: CCOc1ccc([C@@H](N)CF)cn1] | R | 185.1 |
| A2 | 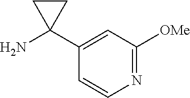 [SMILES: COc1cc(C2(N)CC2)ccn1] | | 165.1 |
| A3 | 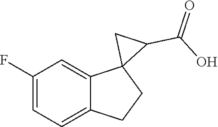 [SMILES: O=C(O)C1CC12CCc1ccc(F)cc12] | Mixture B (cis or trans, 2 diastereomers) | 205.1 [M − H] |
| A4 | 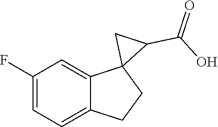 [SMILES: O=C(O)C1CC12CCc1ccc(F)cc12] | Diastereomer C (cis or trans) | 205.1 [M − H] |
| A5 | 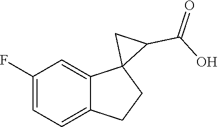 [SMILES: O=C(O)C1CC12CCc1ccc(F)cc12] | Diastereomer D (cis or trans) | 205.1 [M − H] |
| A6 | 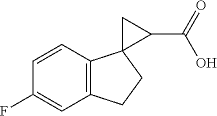 [SMILES: O=C(O)C1CC12CCc1cc(F)ccc12] | Racemic 4 diastereomers | 207.1 |
| A7 | 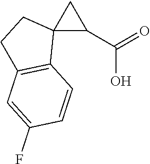 [SMILES: O=C(O)C1CC12CCc1cc(F)ccc12] | Racemic cis | 207.1 |
| A8 | 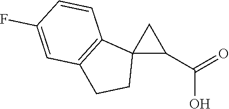 [SMILES: O=C(O)C1CC12CCc1cc(F)ccc12] | Racemic trans | 207.1 |
| A9 | 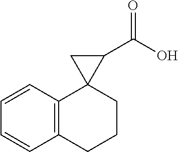 [SMILES: O=C(O)C1CC12CCCc1ccccc12] | Racemic 4 diastereomers | 203.2 |
| A10 | 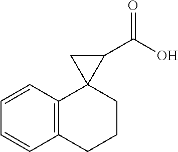 [SMILES: O=C(O)C1CC12CCCc1ccccc12] | Mixture A 2 diastereomers | 203.2 |
| A11 | 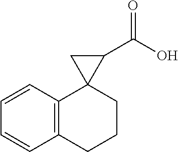 [SMILES: O=C(O)C1CC12CCCc1ccccc12] | Mixture B 2 diastereomers | 203.2 |
| A12 | 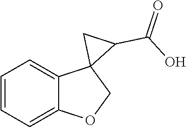 [SMILES: O=C(O)C1CC12COc1ccccc12] | Mixture A 2 diastereomers | 189.1 [M − H] |
| A13 | 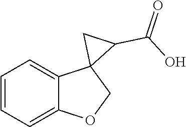 [SMILES: O=C(O)C1CC12COc1ccccc12] | Diastereomer A | 189.1 [M − H] |
| A14 | 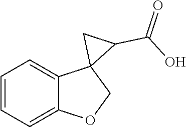 [SMILES: O=C(O)C1CC12COc1ccccc12] | Diastereomer B | 189.1 [M − H] |
| A15 | 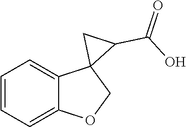 [SMILES: O=C(O)C1CC12COc1ccccc12] | Mixture B 2 diastereomers | 189.1 [M − H] |
| A16 | 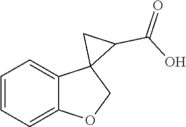 [SMILES: O=C(O)C1CC12COc1ccccc12] | Diastereomer C | 189.1 [M − H] |
| A17 | 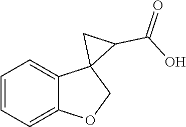 [SMILES: O=C(O)C1CC12COc1ccccc12] | Diastereomer D | 189.1 [M − H] |
| A18 | 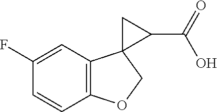 [SMILES: O=C(O)C1CC12COc1ccc(F)cc12] | Racemic 4 diastereomers | 207.1 [M − H] |
| A19 | 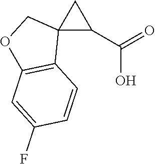 [SMILES: O=C(O)C1CC12COc1cc(F)ccc12] | Racemic trans | 207.1 [M − H] |
| A20 | 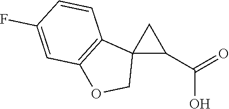 [SMILES: O=C(O)C1CC12COc1cc(F)ccc12] | Racemic cis | 207.1 [M − H] |
| A21 | 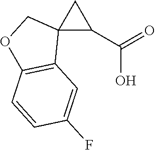 [SMILES: O=C(O)C1CC12COc1ccc(F)cc12] | Trans diastereomer A | 207.1 [M − H] |
| A22 | 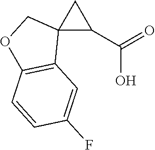 [SMILES: O=C(O)C1CC12COc1ccc(F)cc12] | Trans diastereomer B | 207.1 [M − H] |
| A23 | 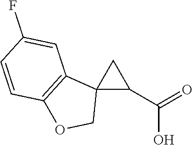 [SMILES: O=C(O)C1CC12COc1ccc(F)cc12] | Cis diastereomer C | 207.1 [M − H] |
| A24 | 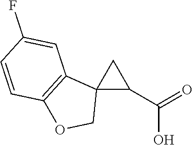 [SMILES: O=C(O)C1CC12COc1ccc(F)cc12] | Cis diastereomer D | 207.1 [M − H] |

### Example 1

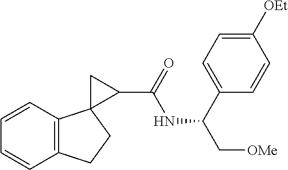 [SMILES: CCOc1ccc([C@H](COC)NC(=O)C2CC23CCc2ccccc23)cc1]

### N-[(1R)-1-(4-Ethoxyphenyl)-2-Methoxy-Ethyl[-2′,3′-Dihydrospiro]Cyclopropane-1,1′-Indene]-2-Carboxamide, Mixture a

To a stirred solution of 2′,3′-dihydrospiro[cyclopropane-1, 1′-indene]-2-carboxylic acid (42 mg. 0.22 mmol) in dichloromethane (S mL) at ambient temperature were added HBTU (82 mg, 0.22 mmol) and N,N-diisopropylethylamine (0.112 mL, 0.66 mmol). After 30 min, (1R)-1-(4-ethoxyphenyl)-2-methoxyethanamine hydrochloride (Intermediate 1) (50 mg, 0.22 mmol) was added and the reaction mixture was stirred at ambient temperature for 18 h. The resulting mixture was concentrated under reduced pressure and the residue was purified by silica gel chromatography, eluting with a gradient of ethyl acetate: hexanes ranging from 0:100 to 50:50. The first pair of diastereomers to elute was N-[ (1R)-1-(4-ethoxyphenyl)-2-methoxy-ethyl]-2′,3′-dihydrospiro[cyclopropane-1, 1′-indene]-2-carboxamide, mixture A, the title compound, and the second pair of diastereomers to elute was N-[(1R)-1-(4-ethoxyphenyl)-2-methoxy-ethyl]-2′,3′-dihydrospiro[cyclopropane-1, 1′-indene]-2-carboxamide, mixture B. MS: m/z=366.3 [M+H]. ¹H NMR (300 MHz, CDCl₃): δ 7.27-7.14 (m, 5H); 6.85 (d, J=8.7 Hz, 2H); 6.71 (m, 1H); 6.21 (d, J=7.5 Hz, 1H); 5.15 (m, 1H); 4.01 (q, J=7.0 Hz, 2H); 3.63-3.54 (m, 2H): 3.32 (s, 3H); 3.05-2.99 (m, 2H): 2.36-2.18 (m, 2H): 1.80 (m, 1H): 1.71 (m, 1H); 1.39 (t, J=7.0 Hz, 3 H); 1.31 (m, 1H).

### Example 2

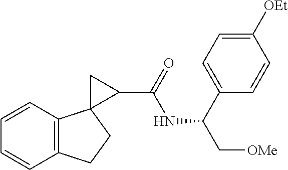 [SMILES: CCOc1ccc([C@H](COC)NC(=O)C2CC23CCc2ccccc23)cc1]

### N-[(1R)-1-(4-Ethoxyphenyl)-2-Methoxy-Ethyl]-2′,3′-Dihydrospiro[Cyclopropane-1,1′-Indene]-2-Carboxamide, Mixture B

The title compound was obtained from the procedure described in Example 1. MS: m/z=366.3 [M+H]. 4H NMR (300 MHZ, CDCl₃): δ 7.25-7.13 (m. 5 H): 6.82 (d, J=8.5 Hz. 2 H); 6.71 (m, 1H): 6.26 (d. J=7.6 Hz, 1H); 5.14 (m, 1H): 4.00 (q. J-7.0 Hz, 2H); 3.63 (m, 2H); 3.34 (s, 3H); 3.05-2.83 (m, 2H); 2.28-2.05 (m, 2H); 1.82 (m, 1H); 1.72 (m, 1H); 1.50-1.32 (m, 4H).

### Example 3

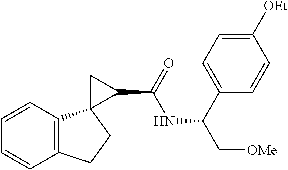

### (1R,2R)-N-[ (1R)-1-(4-Ethoxyphenyl)-2-Methoxy-Ethyl]-2′,3′-Dihydrospiro[Cyclopropane-1,1′-Indene]-2-Carboxamide

To a stirred solution of trans-2′,3′-dihydrospiro[cyclopropane-1,1′-indene]-2-carboxylic acid (Intermediate 8) (135 mg, 0.72 mmol) and (1R)-1-(4-ethoxyphenyl)-2-methoxyethanamine hydrochloride (Intermediate 1) (185 mg, 0.79 mmol) in ethyl acetate (5 mL) at ambient temperature were added N,N-diisopropylethylamine (0.63 mL, 3.59 mmol) and a solution of propylphosphonic anhydride in ethyl acetate (50 wt. %, 0.685 mL, 1.08 mmol) and the reaction mixture was stirred at ambient temperature for 18 h. The resulting mixture was diluted with a saturated aqueous solution of sodium hydrogencarbonate (10 mL) and extracted with ethyl acetate (3×25 mL). The combined organic extracts were washed with a saturated aqueous solution of sodium chloride (10 mL), dried (magnesium sulfate), filtered, and concentrated under reduced pressure. The residue was purified by silica gel chromatography, eluting with a gradient of ethyl acetate: hexanes ranging from 0:100 to 50:50. The first major peak to elute was (1R,2R)-N-[(1R)-1-(4-ethoxyphenyl)-2-methoxy-ethyl]-2′,3′-dihydrospiro[cyclopropane-1, 1′-indene]-2-carboxamide, the title compound, and the second major peak to elute was (1S,2S)-N-[(1R)-1-(4-ethoxyphenyl)-2-methoxy-ethyl]-2′,3′-dihydrospiro[cyclopropane-1, 1′-indene]-2-carboxamide. MS: m z=366.3 [M+H]. H NMR (300 MHz, DMSO-d₆): δ 8.48 (d, J=8.6 Hz, 1H); 7.25-7.10 (m, 5H); 6.90-6.75 (m, 3H); 5.02 (m, 1H); 3.97 (q. J=7.0 Hz, 2H): 3.38 (m, 2H): 3.19 (s, 3H); 3.00-2.80 (m, 2H); 2.20-1.95 (m. 3 H); 1.40 (m, 1H); 1.34-1.18 (m, 4H).

### Example 4

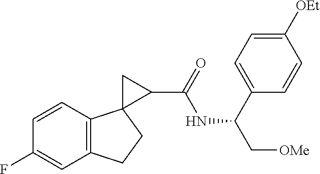 [SMILES: CCOc1ccc([C@H](COC)NC(=O)C2CC23CCc2cc(F)ccc23)cc1]

### Trans-5′-Fluoro-N-[(1R)-1-(4-Ethoxyphenyl)-2-Methoxyethyl]-2′,3′-Dihydrospiro[Cyclopropane-1,1′-Indene]-2-Carboxamide, Diastereomer B

Following procedures analogous to those described in Example 1, but using trans-5-fluoro-2,3-dihydrospiro[cyclopropane-1,1′-indene]-2-carboxylic acid (Intermediate A8) in place of 2′, 3′-dihydrospiro[cyclopropane-1,1′-indene]-2-carboxylic acid, trans-5′-fluoro-N-[(1R)-1-(4-ethoxy phenyl)-2-methoxyethyl]-2′,3′-dihydrospiro[cyclopropane-1, 1′-indene]-2-carboxamide was obtained as a mixture of diastereomers. This mixture was purified by silica gel chromatography, eluting with a gradient of ethyl acetate: hexanes ranging from 0:100 to 50:50. The first major peak to elute was trans-5′-fluoro-N-[(1R)-1-(4-ethoxyphenyl)-2-methoxyethyl]-2′,3′-dihydrospiro[cyclopropane-1, 1′-indene]-2-carboxamide, diastereomer A, and the second major peak to elute was trans-S′-fluoro-N-[ (1R)-1-(4-ethoxyphenyl)-2-methoxyethyl]-2′,3′-dihydrospiro[cyclopropane-1, 1′-indene]-2-carboxamide, diastereomer B, the title compound. MS: m/z=384.2 [M+H]. ¹H NMR (300 MHZ, CDCl₃): δ 7.20 (d, J=8.6 Hz, 2H): 6.90-6.80 (m, 4H); 6.61 (m, 1H): 6.26 (d, J=7.6 Hz, 1H); 5.13 (m, 1H); 4.00 (q. J=7.0 Hz, 2H); 3.63 (m, 2H); 3.34 (s, 3H): 3.03-2.85 (m, 2H): 2.22-2.04 (m, 2H): 1.79-1.67 (m, 2H): 1.41 (t, J=7.0 Hz, 3H): 1.28 (m, 1H).

### Example 5

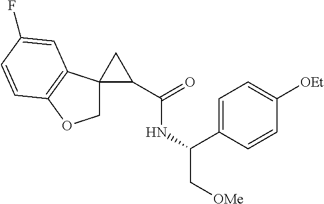 [SMILES: CCOc1ccc([C@H](COC)NC(=O)C2CC23COc2ccc(F)cc23)cc1]

### Cis-5-Fluoro-N-[(1R)-1-(4-Ethoxyphenyl)-2-Methoxyethyl]-2//-Spiro[1-Benzofuran-3,1′-Cyclopropane]-2′-Carboxamide, Diastereomer D

To a stirred solution of cis-5-fluoro-2H-spiro[1-benzofuran-3, 1′-cyclopropane]-2′-carboxylic acid, diastereomer D (Intermediate A24) (45 mg, 0.22 mmol) in dichloromethane (5 mL) at ambient temperature were added HATU (82 mg, 0.22 mmol) and N,N-diisopropylethylamine (0.12 mL, 0.66 mmol). After 30 min, (1R)-1-(4-ethoxyphenyl)-2-methoxyethanamine hydrochloride (Intermediate 1) (50 mg, 0.22 mmol) was added and the reaction mixture was stirred at ambient temperature for 18 h. The resulting mixture was diluted with a saturated aqueous solution of sodium hydrogencarbonate (10 mL) and extracted with ethyl acetate (3×25 mL). The combined organic extracts were washed with a saturated aqueous solution of sodium chloride (10 mL), dried (magnesium sulfate), filtered, and concentrated under reduced pressure. The residue was purified by silica gel chromatography, eluting with a gradient of ethyl acetate: hexanes ranging from 0:100 to 100:0, to afford, after crystallization from diethyl ether, the title compound MS: m/z=386.2 [M+H]. ¹H NMR (300 MHz, CDCl₃): δ 7.21 (d. J=8.6 Hz, 2H); 6.85 (d, J=8.6 Hz, 2H): 6.80 (m, 1H): 6.67 (m, 1H); 6.39 (dd, J=7.9 Hz, 2.7 Hz, 1H): 5.10 (m, 1H): 4.56 (d, J=10.1 Hz, 1H): 4.47 (d. J=10.1 Hz. 1 H); 4.00 (q, J=7.0 Hz, 2H): 3.62 (m, 2H): 3.36 (s. 3 H); 1.95 (m, 1H); 1.73 (m, 1H): 1.45-1.35 (m, 4H).

The examples appearing in the following tables, TABLE EX-A and TABLE EX-B were prepared by analogy to the above examples, as described or prepared as a result of similar transformations with modifications known to those skilled in the art. The requisite starting materials were described herein, commercially available, known in the literature, or readily synthesized by one skilled in the art. Straightforward protecting group strategies were applied in some routes.

TABLE EX-A

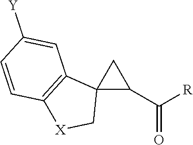

| | | | | Cyclopropane | |
|---|---|---|---|---|---|
| Example | X | Y | R | Stereochemistry | MS [M + H] |
| A1 | CH₂ | H | 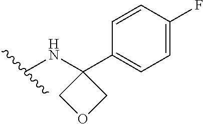 [SMILES: *NC1(c2ccc(F)cc2)COC1] | Racemic 4 diastereomers | 338.3 |
| A2 | CH₂ | F | 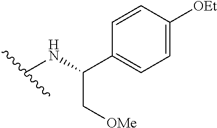 [SMILES: *N[C@@H](COC)c1ccc(OCC)cc1] | Diastereomer A | 384.2 |
| A3 | CH₂ | F | 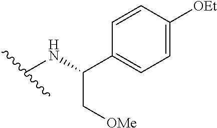 [SMILES: *N[C@@H](COC)c1ccc(OCC)cc1] | Diastereomer B | 384.2 |
| A4 | CH₂ | F | 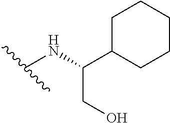 [SMILES: *N[C@@H](CO)C1CCCCC1] | Diastereomer C | 332.3 |
| A5 | CH₂ | F | 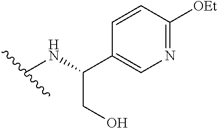 [SMILES: *N[C@@H](CO)c1ccc(OCC)nc1] | Diastereomer C | 371.3 |
| A6 | CH₂ | F | 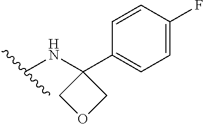 [SMILES: *NC1(c2ccc(F)cc2)COC1] | Diastereomer C | 356.3 |
| A7 | CH₂ | F | 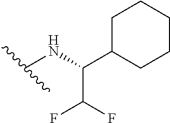 [SMILES: *N[C@@H](C(F)F)C1CCCCC1] | Diastereomer C | 352.3 |
| A8 | O | H | 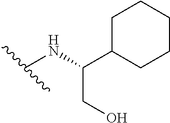 [SMILES: *N[C@@H](CO)C1CCCCC1] | Diastereomer A | 316.3 |
| A9 | O | H | 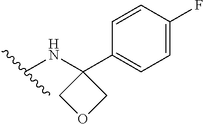 [SMILES: *NC1(c2ccc(F)cc2)COC1] | Diastereomer D | 340.2 |
| A10 | O | F | 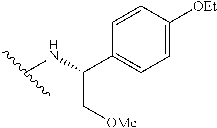 [SMILES: *N[C@@H](COC)c1ccc(OCC)cc1] | Cis diastereomer C | 386.2 |
| A11 | O | F | 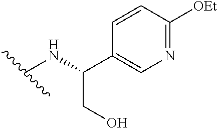 [SMILES: *N[C@@H](CO)c1ccc(OCC)nc1] | Cis diastereomer D | 373.2 |
| A12 | O | F | 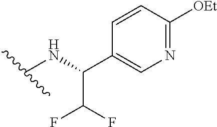 [SMILES: *N[C@H](c1ccc(OCC)nc1)C(F)F] | Cis diastereomer D | 393.3 |
| A13 | O | F | 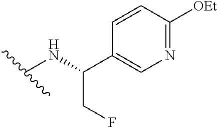 [SMILES: *N[C@@H](CF)c1ccc(OCC)nc1] | Cis diastereomer D | 375.2 |
| A14 | O | F | 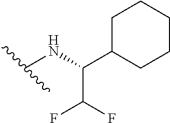 [SMILES: *N[C@@H](C(F)F)C1CCCCC1] | Cis diastereomer D | 354.3 |
| A15 | O | F | 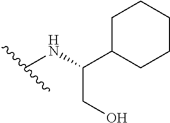 [SMILES: *N[C@@H](CO)C1CCCCC1] | Cis diastereomer D | 334.3 |
| A16 | O | F | 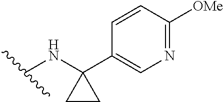 [SMILES: *NC1(c2ccc(OC)nc2)CC1] | Cis diastereomer D | 355.2 |
| A17 | O | F | 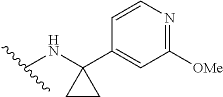 [SMILES: *NC1(c2ccnc(OC)c2)CC1] | Cis diastereomer D | 355.2 |

TABLE EX-B

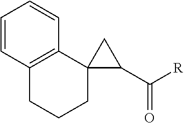

| | | Cyclopropane | |
|---|---|---|---|
| Example | R | Stereochemistry | MS [M + H] |
| B1 | 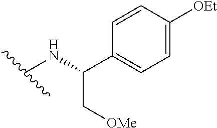 [SMILES: *N[C@@H](COC)c1ccc(OCC)cc1] | Trans diastereomer A | 380.2 |

The utility of the compounds in accordance with the present invention as positive allosteric modulators of a7 nicotinic acetylcholine receptor activity may be demonstrated by methodology known in the art. Direct activation of a7 (agonism), and potentiation of acetylcholine-evoked a7 currents was determined as follows:

Automated patch-clamp electrophysiology was performed using the IonFlux HT (Fluxion Biosciences Inc., San Francisco, CA) in the whole-cell, population patch configuration. Test compounds were assessed for their ability to modulate the function of the a7 nicotinic acetylcholine receptor both in the presence, and in the absence of the natural a7 agonist acetylcholine. A HEK cell line stably expressing both human RIC-3 and human a7 (PrecisION hnAChR a7/RIC-3. Eurofins Pharma, St. Charles, MO) was cultured in 175 cm² triple-layer tissue culture flasks to no more than 90% confluency in DMEM/F-12 growth media supplemented with 10% heat-inactivated fetal bovine serum, 1% non-essential amino acids. 0.625 μg/mL Puromycin, and 400 μg/mL Geneticin. Immediately prior to assay, cells were detached by first aspirating growth media, rinsing with Dulbecco's phosphate buffered saline, and then adding 10 mL of Accutase (Innovative Cell Technologies, San Diego, CA) to the flask and then incubating at 37° C. for 5 minutes. Detached cells were then recovered by the addition of 40 mL of CHO-serum-free media supplemented with 25 mM HEPES, and rocked gently in a 50 ml conical tube for 20 minutes prior to patch-clamp assay. After recovery, cells were pelleted by centrifugation at 1.000 RPM for 1 minute in a compact bench top centrifuge: recovery media was aspirated and cells were resuspended in external recording solution (150 mM NaCl, 5 mM KCl, 2 mM CaCl₂), 1 mM MgCl₂, 10 mM HEPES, 12 mM dextrose) to a density of 5.0×10⁶ cells/mL. The cell suspension was added to the cell inlet wells on an IonFlux HT population patch plate which had previously been rinsed and primed with deionized H₂O. Test compounds were serially diluted in DMSO and then resuspended to the final test concentration in external recording solution, with, or without 40 μM acetylcholine added to the external recording solution: test compounds were then transferred to the IonFlux HT population patch plate. Internal recording solution (110 mM TrisPO₄, 28 mM TrisBase, 0.1 mM CaCl₂), 2 mM MgCl₂, 11 mM EGTA, 4 mM MgATP) was added to the internal recording solution inlet wells on the IonFlux HT patch plate previously loaded with cells and test compounds, and the plate loaded into the lonFlux HT instrument. A protocol was executed on the IonFlux HT to trap the cells, break into the cells, and establish the whole-cell recording configuration: cells were voltage-clamped at a holding potential of −60 mV for the duration of the experiment, all experiments were conducted at room temperature, and the IonFlux HT injection pressure was 8 psi for solution applications. Upon establishing the whole-cell configuration, external recording solution was perfused into the recording chambers for 120 seconds and then 40 μM acetylcholine was applied for 1 second and immediately washed off with external recording solution for 60 seconds. The 40 μM acetylcholine-evoked a7 current served as the current response to which subsequent test compound effects, in the presence, or in the absence of 40 μM acetylcholine would be quantified relative to. Next, test compounds were evaluated at multiple concentrations for their ability to induce, or modulate a7 current responses: three concentrations of test compound were evaluated in ascending dose fashion per recording. To assess test compound agonist activity, test compound diluted in external recording solution was applied starting from the lowest concentration of test compound being tested in the concentration series, for 58 seconds: the first 20 seconds of the 58 second compound application period coincided with a data collection sweep which was 20 seconds in duration, and collected at a rate of 5,000 samples/second. To assess test compound positive allosteric modulator activity, immediately following the 58 second test compound only application period, the same concentration of test compound, diluted in external recording solution containing 40 μM acetylcholine was applied for 1 second: in this way, the test compound and the natural receptor agonist acetylcholine were co-applied, and potentiating effects of test compounds observed. The 1 second application of test compound diluted in external solution containing 40 μM acetylcholine coincided with a data collection sweep which was 20 seconds in duration, and collected at a rate of 5,000 samples/second, after which, external recording solution only was applied for 42 seconds. Following this 42 second wash with external recording solution only, the next highest concentration of the test compound in the concentration series was applied in the absence and then in the presence of acetylcholine as previously described, and data collected as previously described. After test compound agonist, and positive allosteric modulator activity were assessed at three ascending concentrations, the experiment was terminated and leak subtraction performed using the lonFlux HT data analysis software. Peak current amplitudes and the area under the curve (AUC) were both quantified for each current sweep using proprietary software and test compound effects where quantified as follows.

Test Compound Agonist Activity was Calculated as:

$$\% Agonism = ( Y / X ) \times 100$$

Test compound potentiator activity was calculated as:

$$\% Potentiation = [ ( Z / X ) \times 100 ] - 100$$

X=Peak current amplitude (or AUC) evoked by 40 μM acetylcholine
Y=Peak current amplitude (or AUC) evoked by test compound diluted in external recording solution
Z=Peak current amplitude (or AUC) evoked by test compound diluted in external recording solution containing 40 UM acetylcholine

As such, test compounds which evoked the same current amplitude as 40 μM acetylcholine alone would exhibit a calculated % Agonism of 100%. Test compounds co-applied with 40 UM acetylcholine which evoked a current amplitude 2× the current evoked from 40 μM acetylcholine alone would exhibit a calculated % Potentiation of 100%, whereas test compounds co-applied with 40 μM acetylcholine which evoked the same current amplitude as 40 μM acetylcholine alone would be characterized as exhibiting no potentiation.

Agonist and potentiation data, derived by peak current amplitude or area under the curve (AUC) were graphed and fit using a 4-parameter logistic fit based on the Levenberg-Marquardt algorithm where y=A+ ((B-A)/(1+((C/x)\hat{ }D))) where:
A=Minimum
B=Maximum
C=EC₅₀
D=Slope
x=test compound concentration
y=% Agonism or % Potentiation

Potency data for selected compounds of the present invention in the automated patch-clamp electrophysiology functional assay (Assay A) are represented in the table below.

Table
POTENCY DATA FOR SELECTED COMPOUNDS

| | α7 nAChR |
|---|---|
| Example | Potency* |
| 1 | C |
| 2 | B |
| 3 | C |
| 4 | C |
| 5 | B |
| A1 | C |
| A2 | C |
| A3 | C |
| A4 | C |
| A5 | C |
| A6 | C |
| A7 | C |
| A8 | C |
| A9 | C |
| A10 | C |
| A11 | D |
| A12 | C |
| A13 | C |
| A14 | C |
| A15 | C |
| A16 | C |
| A17 | C |
| B1 | C |

*Potency defined as A (EC₅₀ ≤ 0.1 μM); B (0.1 μM < EC₅₀ ≤ 0.5 μM); C (0.5 μM < EC₅₀ ≤ 5 μM); D (5 μM < EC₅₀ ≤ 50 μM)

Electrophysiology EC₅₀ values for selected compounds of the present invention in the automated patch-clamp electrophysiology functional assay (Assay A) are provided in the table below:

TABLE: ELECTROPHYSIOLOGY EC₅₀ VALUES FOR
SELECTED COMPOUNDS

| | a7 nAChR |
|---|---|
| Example | EC₅₀ (nM) |
| 1 | 4500 |
| 2 | 420 |
| 3 | 690 |
| 4 | 590 |
| 5 | 480 |
| A1 | 3200 |
| A2 | 1600 |
| A3 | 700 |
| A4 | 1200 |
| A5 | 1400 |
| A6 | 820 |
| A8 | 630 |
| A9 | 1100 |
| A11 | 5800 |
| A12 | 3000 |
| A13 | 790 |
| A15 | 770 |
| A17 | 2300 |
| B1 | 740 |

It will be appreciated that various of the above-discussed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A compound having the formula I:
relates to a compound of formula I: 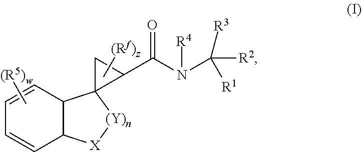
or a pharmaceutically acceptable salt thereof, wherein;
w is 0, 1, 2, 3, or 4;
n is 1, 2, 3, or 4;
z is 0, 1, 2, or 3;
q is 0, 1, or 2;
X is O, C(R^{d}R^{e}), NH, or S(O) m;
Y is C(R′R);
R^{d} and R^{e} are each independently selected from hydrogen, halogen, (C₁-C₄)alkyl and (C₁-C₄)haloalkyl;
each R^{f}, R^{e} and R^{h} is independently selected from halogen, (C₁-C₄)alkyl and (C₁-C₄)haloalkyl;
m is 0, 1, or 2;
each R⁵ is independently selected from halogen, cyano, hydroxy, (C₁-C₄)alkyl, (C₃-C₆)cycloalkyl, (C₁-C₄)alkoxy, aryl, heteroaryl, and heterocyclyl, wherein said alkyl, cycloalkyl, aryl, heteroaryl and heterocyclyl are optionally substituted with one or more substituents independently selected from R⁶;
R⁶ is halogen, OH, (C₁-C₄)alkyl, aryl, heteroaryl, or heterocyclyl;
R⁴ is hydrogen or (C₁-C₄)alkyl;
R¹ is hydrogen, (C₁-C₄)alkyl or (C₁-C₄) heteroalkyl;
R² and R³ are each independently selected from hydrogen, hydroxy, oxo, halogen, cyano, (C₁-C₈)alkyl, S(O)_{q}(C₁-C₆)alkyl, (C₁-C₈)haloalkyl, (C₁-C₄) heteroalkyl, (C₁-C₆) alkoxy, (C₁-C₆)alkoxy (C₁-C₆)alkyl, (C₁-C₆)alkylamino, amino (C₁-C₆)alkyl, hydroxy (C₁-C₆)alkyl, (C₃-C₈)cycloalkyl, (C₃-C₈)cycloalkyl(C₁-C₆) alkoxy, heterocyclyl, aryl, and heteroaryl; wherein said alkoxy, alkylamino, aminoalkyl, hydroxyalkyl, alkyl, haloalkyl, cycloalkyl, aryl, heteroaryl and heterocyclyl are optionally substituted with one or more R⁷ substituents;
each R⁷ substituent is independently selected from F, Cl, Br, OH, oxo, CF₃, OCF₃, CN, (C₁-C₆)alkyl, O(C₁-C₄)alkyl, S(C₁-C₄)alkyl, C═O(C₁-C₄)alkyl, (C═O) NR8R′, (C═O) OR⁸, (C₁-C₄)alkynyl, (C₃-C₆)cycloalkyl, O(C₃-C₆)cycloalkyl, C═O(C₃-C₆)cycloalkyl, aryl, heteroaryl and heterocyclyl, wherein said alkyl, aryl, heteroaryl and heterocyclyl are independently substituted with 0, 1, 2, 3, or 4 R¹⁰ substituents; or
when R¹ is (C₁-C₄)alkyl and R² is (C₁-C₈)alkyl(C₁-C₄) heteroalkyl, or (C₁-C₅)haloalkyl, R¹ and R² together with the carbon atom to which they are attached, optionally may form a 3- to 8—membered unsaturated ring substituted with one or more R⁷ substituents;
R⁸ is H or (C₁-C₄)alkyl;
R⁴ is H or (C₁-C₄)alkyl; and
R¹⁰ is halogen, (C₁-C₄)alkyl, CF₃, OH or oxo.

2. The compound of claim 1, or a pharmaceutically acceptable salt thereof, wherein R¹ is hydrogen or (C₁-C₄)alkyl.

3. The compound of claim 1, or a pharmaceutically acceptable salt thereof, wherein R² and R³ are each independently selected from hydroxy, halogen, (C₁-C₈)alkyl, S(O)_{q}(C₁-C₆)alkyl, (C₁-C₈)haloalkyl, (C₁-C₄) heteroalkyl, (C₁-C₆) alkoxy, (C₁-C₆)alkoxy (C₁-C₆)alkyl, (C₁-C₆)alkylamino, amino (C₁-C₆)alkyl, hydroxy (C₁-C₆)alkyl, (C₃-C₈)cycloalkyl, (C₃-C₈)cycloalkyl(C₁-C₆) alkoxy, heterocyclyl, aryl, and heteroaryl; wherein said alkoxy, alkylamino, aminoalkyl, hydroxyalkyl, alkyl, haloalkyl, cycloalkyl, aryl, heteroaryl and heterocyclyl are optionally substituted with one or more R⁷ substituents.

4. The compound claim 1, or a pharmaceutically acceptable salt thereof, wherein R¹ is (C₁-C₄)alkyl and R² is (C₁-C₈)alkyl(C₁-C₄) heteroalkyl, or (C₁-C₅)haloalkyl and R¹ and R² together with the carbon atom to which they are attached, form a 3- to 8—membered unsaturated ring substituted with one or more R⁷ substituents.

5. The compound of claim 3, or a pharmaceutically acceptable salt thereof, wherein X is O or C(R^{d}R^{e}).

6. The compound of claim 5, or a pharmaceutically acceptable salt thereof, wherein each R^{f} and R^{e} is independently selected from halogen, methyl, ethyl, propyl, trifluormethyl, trifluoroethyl, difluoromethyl.

7. The compound of claim 6, or a pharmaceutically acceptable salt thereof, wherein each R⁵ is independently selected from halogen, cyano, hydroxy, (C₁-C₄)alkyl, and (C₁-C₄)alkoxy, wherein said alkyl is optionally substituted with one or more substituents independently selected from R⁶.

8. The compound of claim 7, or a pharmaceutically acceptable salt thereof, wherein each R⁷ substituent is independently selected from F, C₁, OH, oxo, CF₃, OCF₃, CN, (C₁-C₆)alkyl, O(C₁-C₄)alkyl, (C₃-C₆)cycloalkyl, O(C₃-C₆)cycloalkyl, C═O(C₃-C₆)cycloalkyl, aryl, heteroaryl and heterocyclyl, wherein said alkyl, aryl, heteroaryl and heterocyclyl are independently substituted with 0, 1, 2, 3, or 4 R¹⁰ substituents.

9. The compound of claim 8, or a pharmaceutically acceptable salt thereof, wherein each R^{d} and R^{e} is independently selected from hydrogen, halogen, methyl, ethyl, propyl, trifluormethyl, trifluoroethyl, and difluoromethyl.

10. The compound claim 9, or a pharmaceutically acceptable salt thereof, wherein R^{e} is hydrogen.

11. The compound of claim 10, or a pharmaceutically acceptable salt thereof, wherein n is 1 or 2.

12. The compound of claim 11, or a pharmaceutically acceptable salt thereof, wherein w is 0, 1 or 2.

13. The compound of claim 1 which is selected from 1-({[2-(difluoromethoxy)pyridin-4-yl]oxy}methyl)-6-azaspiro[2.5]octane-6-sulfonamide; or a pharmaceutically acceptable salt thereof.

14. A pharmaceutical composition comprising (i) a pharmaceutically acceptable carrier and (ii) a compound of claim 1 or a pharmaceutically acceptable salt thereof.

15. The pharmaceutical composition of claim 13, further comprising a second therapeutic agent selected from the group consisting of acetylcholinesterase inhibitors; NMDA receptor antagonists; antipsychotics; MAO-B inhibitors; and levodopa.

16. (canceled)

17. A method of modulating a7 nAChR activity in a patient in need thereof, the method comprising administering to the patient the compound of claim 1 or a pharmaceutically acceptable salt thereof, in an amount effective to modulate a7 nAChR activity.

18. (canceled)

19. A method of treating a patient with cognitive impairments associated with Alzheimer's disease, Parkinson's disease, and schizophrenia, the method comprising administering to the patient the compound of claim 1, or a pharmaceutically acceptable salt thereof, in an amount effective to treat the patient.
